(19) United States
(12) Patent Application Publication
(10) Pub. No.: US 2018/0247711 A1
(43) Pub. Date: Aug. 30, 2018

(54) SYSTEM AND METHOD FOR MANAGING THE USE OF SURGICAL INSTRUMENTS

(71) Applicant: Applied Logic, Inc., Saint Louis, MO (US)

(72) Inventor: William Michael TERRY, Chesterfield, MO (US)

(73) Assignee: Applied Logic, Inc.

(21) Appl. No.: 15/903,627

(22) Filed: Feb. 23, 2018

Related U.S. Application Data
(60) Provisional application No. 62/464,080, filed on Feb. 27, 2017.

(51) Int. Cl. G16H 40/40; G06K 19/07; A61B 50/33; A61B 90/98

(52) U.S. Cl. CPC G16H 40/40; G06K 19/0723; G06K 7/10009; A61B 90/98; A61B 50/33

(57) ABSTRACT

Disclosed herein are systems and methods for managing surgical instruments. The method includes storing expected processing times associated with managing a group of surgical instrument trays being processed through a sterilization processing department, the expected processing times comprising at least a first expected processing time associated with the first portion of processing the group of surgical instrument trays through the department and a second expected processing time associated with the second portion of processing the group of surgical instrument trays, tracking, in real-time, the group of surgical instrument trays through the sterilization processing department and comparing the time-based data to the expected processing times to identify a discrepancy associated with a respective surgical instrument tray of the group of surgical instrument trays. The method includes identifying metadata associated with the respective surgical instrument tray and transmitting an alert to a device based on the discrepancy and the metadata.

### PRIORITY

This application claims priority to U.S. Provisional Patent Application No. 62/464,080 filed 27 Feb. 2017, which is herein incorporated by reference in its entirety.

### BACKGROUND

### 1. Technical Field

The present disclosure relates to various computing devices, computing management systems, and computing environments that automate the management of surgical instruments and surgical assets.

### 2. Introduction

The delivery of surgical instrumentation of hospitals today has become complex and expensive. Hospitals have moved towards automation and digitalization of the sterile processing department. These efforts are at the forefront of patient care. FIG. 1 illustrates the general process of managing surgical assets in hospital 100. A tray or group of surgical instruments or assets 104 is provided in a hospital room 102 in order to provide care for the patient 120. Various staff members including nurses 116 and doctors 118 utilize the instruments 104 for surgical procedures or other procedures. Following surgery, the surgical instruments must be moved 106 to a sterile processing department 122 for cleaning, sterilization, and preparation for additional surgeries. Computer systems can be provided which can automate the management of such surgical inventory.

As a general example, staff 126 in the sterile processing department will move instrument trays 108, 109 through a cleaning process which and can include decontamination as well sterilization using a sterilization unit 110. The trays have barcodes which can identify the trays by name, number and other identifying data. Equipment used for processing the trays includes barcode readers which can be used to track the progress of each particular respective instrument tray. Trays 112 and 114 are represented as being within a sterilization unit 110. Once the instrument trays are sterilized, they are in a state 112 of being ready to be moved 114 to an operating room 102 for use. Other staff members of the hospital 112, 124 will play a role in managing the process or will need to know the status of individual trays that may be associated with surgeries that are scheduled.

There are many challenges in this overall process that are common in patient care. Instrument trays may be missing a particular surgical instrument. The timing of the availability of a complete instrument tray that is sterile and prepared to use is critical for patient care. When the patient goes under anesthesia, the surgeon may discover that a critical instrument is missing from the instrument tray and needs to be found and perhaps sterilized separately. The delay to accomplish this task can cause damage to the patient who should only be under anesthesia for short amount of time or who has only been given a certain amount of anesthesia for a planned period of time for the surgery. In another aspect, the surgeon may use an alternate instrument or seek to have a known soiled instrument quickly cleaned in the operating room. Every time an operating room door is opened or a wound is left open to the air for longer than necessary, patient care can suffer. Infections can increase in these scenarios.

Conventional surgical inventory management computing systems, sterilization systems, and/or the like, are insufficient for providing immediate response (e.g., real-time) information and ensuring the level of accuracy and quality of surgical instrumentation that is often required to prevent infection and harm to patients. Stated differently, there is a lack of an ability to communicate issues surrounding the management and sterilization of surgical instruments in real-time, which can lead to delays in patient care. For example, there is a lack of coordination between existing medical inventory computing systems and surgical asset management systems. Conventional surgical instrument computing systems cannot automatically connect and communicate with other types of medical-related computing systems and environments, medical devices, medical software, and/or the like, any of which may be used by hospitals and physicians during the care and treatment of a patient. Additionally, conventional surgical asset management systems do not employ standardized data mechanisms (e.g., the use of metadata) to represent surgical inventory assets, causing the devices to be incompatible with commonly-used and standardized Computer-assisted Surgery Systems (“CAS”), Computerized Maintenance Management Systems (“CMMS”), instrument tracking software, among others.

Accordingly, what is needed is an improved computing system and networking environment that can automatically implement and execute a process for managing the cycle for the use of surgical assets.

### SUMMARY

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Disclosed are systems, methods, and non-transitory computer-readable storage media for improving the management of surgical assets by coordinating the use of surgical inventory such that operating room and sterile processing department teams are connected in such a way that enables appropriate real-time alerts, such as mobile alerts, and team messaging right on mobile devices. The inventory management system disclosed herein can reduce error, increase productivity, increase the quality of the use of surgical instrumentation, and increase the accountability of individual people responsible. The ultimate goal is the timely delivery of complete, sterile, and on-time surgical instrumentation that will reduce delays in case start times, nursing time spent on non-patient functions and surgical complaints related to incomplete surgical sets. The disclosure herein coordinates in a novel way inventory, operating room equipment and/or personnel and sterile processing department (SPD) systems, personnel, and surgical instruments. The system can improve the process by bringing the OR and SPD teams together through trade tracking, auto alerts, direct messaging and real-time inventory data. An application is provided on mobile devices that connects and communicates real-time alerts to key players when issues arise. Customized banners can be provided which broadcast custom hospital information in inventory updates to all necessary users. A messaging component maintains and documents communication from SPD managers and their staff. Furthermore, system dashboards can be provided which present dynamic and real-time monitoring of department operations, enable easy to use and drill down layouts and turn data into actionable knowledge.

A system “focus” feature can provide specific directions to a member of an SPD team to assemble a critical instrument tray and prioritize the SFP team's minute to minute production which ultimately leads to improved surgical timing and experience.

Various embodiments or aspects of this disclosure can be claimed from different perspectives. For example, one embodiment could focus on the application that is implemented on various mobile devices of users. The functionality that is performed by the application can be the subject of one embodiment. In another aspect, a server will receive data about the real-time monitoring of the processing of surgical instrument trays and provide notifications as disclosed herein. Thus, another embodiment could be the processing from the standpoint of the server.

The disclosed system uses end-user application settings to correlate with metadata held in the system that when combined and processed, provides intelligent direction on which alerts are applicable to which end users.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 illustrates an example hospital with an operating room and a sterile processing department;

FIG. 2 illustrates a functional block diagram that illustrates an exemplary system embodiment;

FIG. 3A illustrates an exemplary system for surgical asset management;

FIG. 3B illustrates use of RFID technology;

FIG. 3C illustrates a multi-hospital context;

FIG. 4 illustrates an example application interface;

FIG. 5 illustrates another example application interface with various notices; and

FIG. 6 illustrates an example “focus” notification of an action item to be taken by an individual;

FIG. 7 illustrates a messaging embodiment;

FIG. 8 illustrates a biological indicator messaging embodiment;

FIG. 9 illustrates a dashboard view related to inventory processed;

FIG. 10 illustrates a dashboard view of loaner instruments checked in;

FIG. 11 illustrates a dashboard view of a status of instrument inventory;

FIG. 12 illustrates a dashboard view of tray performance;

FIG. 13 illustrates a dashboard view of top instruments missing;

FIG. 14 illustrates a dashboard view of trays assembled;

FIG. 15 illustrates a dashboard view of minutes assembling trays;

FIG. 16 illustrates a dashboard view of unused trays in storage;

FIG. 17 illustrates a dashboard view of unused instruments in storage;

FIG. 18 illustrates a dashboard view of lost instruments;

FIG. 19 illustrates a dashboard view of number of instruments lost by specialty;

FIG. 20 illustrates a dashboard view of top instruments lost;

FIG. 21 illustrates a dashboard view of inventory not sterile;

FIG. 22 illustrates a dashboard view of inventory sent out but not returned;

FIG. 23 illustrates the use of RFID technology in a method embodiment;

FIG. 24 illustrates an intelligence scheduling method embodiment;

FIG. 25 illustrates the use of a global ID according to a method embodiment; and

FIG. 26 illustrates a method embodiment related to multihospital management.

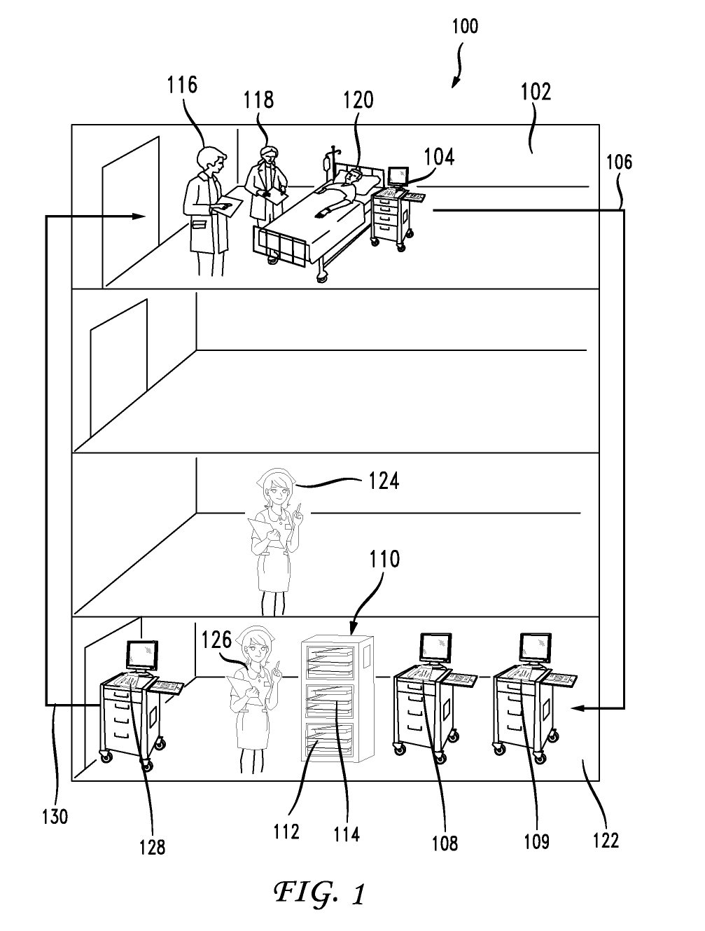

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

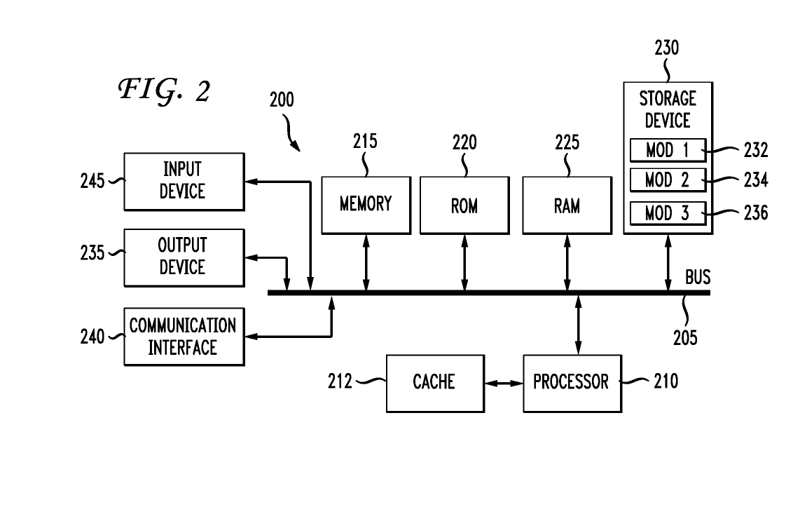

The present disclosure addresses the need identified above in the art of managing the cycle of use of surgical instruments. Systems, methods and non-transitory computer-readable media are disclosed which describe improvements in terms of one or more of hardware, software, algorithms, processes, and so forth. A brief introductory description of a basic general purpose system or computing device in FIG. 2 which can be employed to practice the concepts is disclosed herein. A more detailed description of specific improvements then follow, with variations and embodiments set forth. The disclosure now turns to FIG. 2.

The disclosure next turns to FIG. 2 which generally describes a computer, such as a computer client or server that may be employed to automate the management of surgical instruments and assets. FIG. 2 illustrates a computing system architecture 200 wherein the components of the system are in electrical communication with each other using a bus 205. Exemplary system 200 includes a processing unit (CPU or processor) 210 and a system bus 205 that couples various system components including the system memory 215, such as read only memory (ROM) 220 and random access memory (RAM) 225, to the processor 210. The system 200 can include a cache of high-speed memory connected directly with, in close proximity to, or integrated as part of the processor 210. The system 200 can copy data from the memory 215 and/or the storage device 230 to the cache 212 for quick access by the processor 210. In this way, the cache can provide a performance boost that avoids processor 210 delays while waiting for data. These and other modules can control or be configured to control the processor 210 to perform various actions. Other system memory 215 may be available for use as well. The memory 215 can include multiple different types of memory with different performance characteristics. The processor 210 can include any general purpose processor and a hardware module or software module, such as module 1 232, module 2 234, and module 3 236 stored in storage device 230, configured to control the processor 210 as well as a special-purpose processor where software instructions are incorporated into the actual processor design. The processor 210 may essentially be a completely self-contained computing system, containing multiple cores or processors, a bus, memory controller, cache, etc. A multi-core processor may be symmetric or asymmetric.

To enable user interaction with the computing device 200, an input device 245 can represent any number of input mechanisms, such as a microphone for speech, a touch-sensitive screen for gesture or graphical input, keyboard, mouse, motion input, speech and so forth. An output device 235 can also be one or more of a number of output mechanisms known to those of skill in the art. In some instances, multimodal systems can enable a user to provide multiple types of input to communicate with the computing device 200. The communications interface 240 can generally govern and manage the user input and system output. There is no restriction on operating on any particular hardware arrangement and therefore the basic features here may easily be substituted for improved hardware or firmware arrangements as they are developed.

Storage device 230 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 225, read only memory (ROM) 220, and hybrids thereof.

The storage device 230 can include software modules 232, 234, 236 for controlling the processor 210. Other hardware or software modules are contemplated. The storage device 230 can be connected to the system bus 205. In one aspect, a hardware module that performs a particular function can include the software component stored in a computer-readable medium in connection with the necessary hardware components, such as the processor 210, bus 205, display 235, and so forth, to carry out the function.

The present disclosure relates to improved structures and methods for using a pressure sensitive button or screen to control media. The principles set forth herein can also be used to control any functionality, device, vehicle, remote control cars, appliances, security systems, and so forth. Most of the disclosure will relate to a particular features relating to media, but where applicable, the principles can apply to any control operation.

Various aspects or features will be disclosed herein. For example, a messaging application will be introduced, an “app” which can be installed on individual user mobile devices is also introduced which will connect with the system to be able to provide the necessary user interface and user interaction to be able to receive messages, notices, access a dashboard of information, initiate communication, and so forth. Another aspect involves biological indicator which can confirm success in the sterilization process. The technology associated with providing necessary notices and information related to use of the biological indicators is also provided. Another aspect of this disclosure relates to RFID tracking. New computer infrastructure can be provided in the overall network disclosed herein such that individual instrument trays as well as staff members can have RFID's which are detected at various specific locations within a hospital or sterilization processing department in order to obtain additional layers of information that can help the algorithms disclosed herein to provide the necessary notice and communications to improve the overall process. Another aspect of this disclosure relates to the intelligence scheduling of surgeries using the inventory processing data. A novel features disclosed herein relates to utilizing the information obtained through managing surgical instrument tray processing such that algorithms can be utilized to provide suggestions or direct scheduling of surgical procedures based on the knowledge that the system has with respect to the availability of the surgical instruments necessary for that procedure. In another aspect, disclosed herein will be a multi-hospital management approach in which the principles are applied to multiple hospitals. Given this extra layer of complexity, additional novelties are provided which enables improved management of surgical instrument trays across a multihospital environment. Finally, an approach is disclosed with respect to establishing a global identification number for surgical instrument trays which is unique to that particular tray across the region which can include a city, state, a nation, or even the world. Centralized management of the Global ID for each individual instrument tray can be provided which can improve the overall management of surgical instrument trays, particularly with respect to the use of loaner trays which can be delivered from one hospital to another. These various improvements to the overall process of managing the use, scheduling and sterilization of surgical instrument trays becomes possible utilizing the computer infrastructure disclosed herein. It is noted that no individual embodiment is necessarily required in this disclosure. Any individual concept or idea can be mixed and matched with any other concepts disclosed herein to arrive at a particular claimed feature. Thus, while different aspects or embodiments are discussed for the purpose of explaining the principles disclosed herein, each should be considered an example of various features which can be blended with or mixed with any other features discussed in any other “embodiment”.

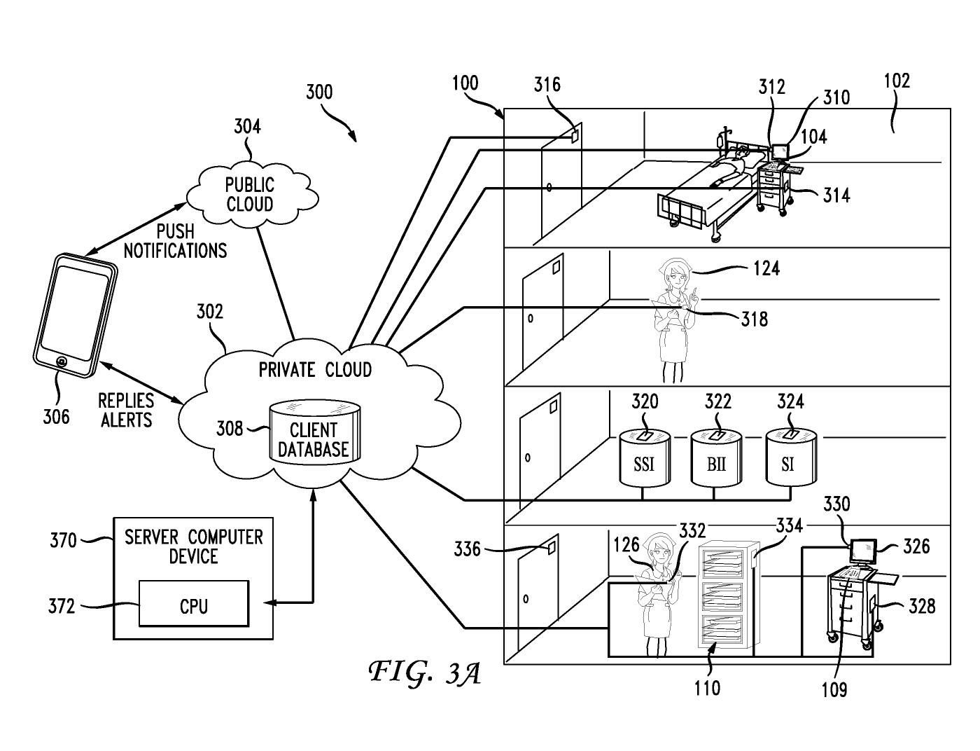

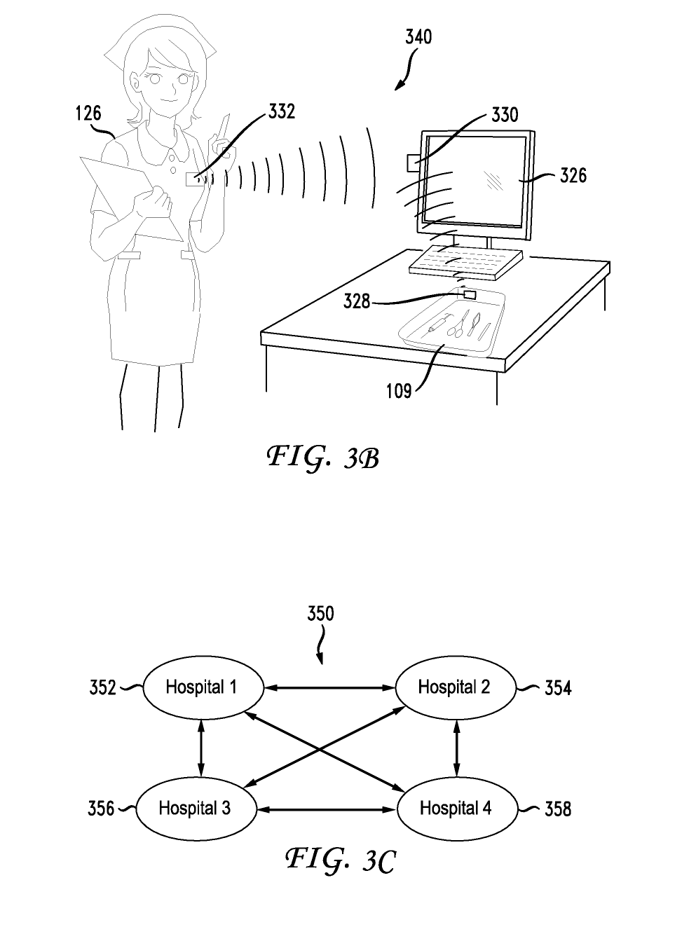

FIG. 3A illustrates an example computing network or environment 300 that can be used in a hospital setting to automatically track and manage surgical instruments and assets. In the illustrated embodiment, a private cloud 302 is employed that can communicate with various computing devices and/or medical devices contained within the hospital 100 or otherwise associated with the hospital 100.

In one specific example, the private cloud 302 may communicate with the public cloud 304 to provide notifications messages and other data which can then be pushed to client devices 306. Client device 306 can be an iPhone, Android device, desktop device, laptop device, or any other type of processing device and/or computing device with which a user can interact to receive messages, notifications, and/or the like. A user may interact with the client device 306 to provide replies or read alerts and generally communicate data with the private cloud 302. The client device 306 can store an application that enables the communication of data associated with surgical instrument management. For example, the client device may receive data from and transmit data to the various devices of the hospital 100.

The hospital 100 is shown by way of example to have various computing technologies and computing devices that communicate one with another. For example, operating room 102 can include a computer 310 having an RFID 312 or a radiofrequency identification unit. In one aspect, surgical trays, computing devices, staff, sterilizations machines, and so forth can each have a respective RFID which can be used to communicate and coordinate information with respect to surgical asset management. In the illustrated embodiment, instrument tray 104 in the surgical room 102 is shown as having an RFID 314. A doorway or a wall in the surgical room 102 can also have an RFID reader or detector 316. Staff member 122 can wear an RFID 318 as well. Various databases for surgical scheduling interface (SSI) 320, biological indicator interface (BII) 322 and sterilizer interface (SI) 324 may communicate with a client database 308 for a particular hospital included in the private cloud 302. In the sterilization processing department 122, an instrument tray 109 can have its own RFID 328, computing devices 326 can include RFID readers 330, staff 126 can have an RFID 332, a sterilization unit 110 can have an RFID reader 334 and a doorway or wall can have an RFID reader 336. In this technical environment, those people or entities that have RFIDs can be detected or read by RFID readers at the various locations in the overall system, such as on the sterilization unit 110, a computing device or doorway. The information about the location of people and/or trays of instruments or other components can be coordinated at a central computer system for managing the instrument trays according to the principles disclosed herein.

In one specific example, the computing network/environment 300 can include a server computing device 370 that executes various algorithms or otherwise provides specific functionality to automatically manage the surgical assets of the hospital 100, coordinates communication between the various devices of the hospital 100 and provides triggered alerts to one or more of the client devices 306 at appropriate times when asset management issues arise. For example, algorithms may be executed to evaluate the data that is received through tracking one or more of instrument trays and particular individuals in the process, as well as particular gap times which relate to expected time frames at various stages of the process of preparing through sterilization and other processes instrument trays for surgery, in order to in real time trigger a message or a notice to one or more mobile devices associated with one or more individuals that can be specifically related to a particular instrument tray. Depending on the analysis performed by the algorithm, the system will automatically initiate the proper functionality to ensure that a particular instrument tray will be processed properly in preparation for surgery. The functionality may be a message or messages sent to individual staff members. The functionality may be a “focus” notice to an individual in the SPD was responsible for that particular instrument tray with instructions to perform a specific task. The functionality can include preparing a specifically tailored message to a nurse with selectable objects, such as buttons, which can implement specific instructions. For example, a communication can be tailored to the nurse with one button which can be selected to simply accept the fact that there may be an issue with sterilization of the surgical tray for the upcoming procedure. Another button can be configured to be selectable by the nurse to send a focus message to John Smith in the SPD to separately sterilize that tray and bring it immediately to the operating room. Other selectable objects could also be configured and presented in the tailored message. The method in this regard will involve utilizing the data regarding staff members, processing of instrument trays, a particular surgical procedure and needs, as well as any other data or parameters disclosed herein to generate a tailored message or interface which can simplify the options that are selectable to be those that would particularly relate to the processing of the respective instrument tray for this particular surgery. Although the server computing device 370 is illustrated as being located outside of the private cloud 308, it is contemplated that it may be located within the private cloud 308, or elsewhere in the overall environment 300, and may communicate with the devices of the network directly or remotely.

FIG. 3B illustrates the use 340 of RFID technology according to the present disclosure. An RFID mentioned above can also include an RFID reader. For example, device 310 or device 326, or sterilization unit 110, or RFID's 330, 316, 336 can be RFID readers such that if a particular user 126 is standing in front of the sterilization unit 110 inputting instrument trays for sterilization, the RFID reader 334 can identify that particular individual from the RFID data of the RFID associated with the user 126. The identified individual can be identified also at a particular computer station 326.

Generally, RFID technology is known to include small electronic devices that have a small chip and antenna and that can carry a particular amount of data. The RFID can provide a unique identifier for an individual object or person. The RFID technology can work typically within a few feet or up to 20 feet for higher frequency devices. Thus, RFID's can be scanned by this scanner RFID's typically within such a range as is illustrated in FIG. 3B.

Another aspect of this process is loaner instrument trays. The instruments are very expensive and many hospitals will schedule surgical procedures and request loaner trays from another hospital or facility. The hospital that is performing the surgery, however, is responsible for receiving the loaner tray and sterilizing it in preparation for the surgery. A common problem with respect to timing is that the loaner surgical tray is received at the hospital performing the surgery without sufficient time for sterilization. For example, if the hospital receives the tray five hours before the surgery is scheduled, then the normal flow of inventory in the SPD 122 may not allow sufficient time for it to be properly sterilized. The system disclosed herein will track and monitor loaner trays. For example, a doctor or a staff member can indicate that they desire alerts for loaner trays delivered late to the hospital. Thus, the server computing device 370 may automatically generate a notification identifying a loaner tray as being received late and automatically transmit the notification to the client device 306 or one of the other computing devices and/or medical devices located within the hospital 100 for use and access by a user, such as the user 126. The notifications provided by the server computing device 370 may embed, encapsulate, or otherwise encode various pieces of data and/or information and/or metadata within the notifications that may be used to uniquely identify the loaner tray or other surgical assets. Generally speaking, metadata is data that describes how and when and by what device a particular set of data was collected or it could include any parameter or data disclosed herein related to processing instrument trays. Thus, in the present application, metadata refers to data that may be used to describe and/or identify portions of the data obtained by the server computing device 370 and/or any of the other various devices of the computing environment 300 during the management of surgical instruments and surgical assets. For example, such information may include a representative's name, a manufacturer's name for the surgical instruments, a time of delivery, individuals who prepared the tray, and so forth. Thus, if the hospital requires a certain time period for the tray to arrive prior to surgery, if that time period is not satisfied, a notification may be generated by the server computing device 370 and automatically transmitted to the client device 306 and/or the various computing devices and medical devices of the hospital 100. Loaner tray can be identified through a barcode with a barcode reader or through an RFID which is previously associated with the loaner tray or attached to the loaner tray as arise at the borrowing hospital. It is noted that barcodes can be associated with trays which can be scanned to track the progress of an individual tray through the process. There are laws that required documenting all of the instruments used on the patient and their treatment. The system disclosed herein improves the process and provides a properly documented and signed electronic record. Thus, whether the system uses barcodes or the additional technology of RFIDs, the legal requirements of tracking the progress of individual trays can be complied with.

An illustrative example of the server computing device 370 generating a time-based notification or alert will now be provided. Assume a loaner tray must be received at the hospital 100 at least 36 hours before surgery to ensure there is enough time to perform proper sterilization. If the tray is delivered 20 hours before a surgery, an alert can be generated automatically by the computer server device 370 and automatically transmitted to the various devices of the hospital 100. The alert can be particularly configured with selectable objects that are specifically related to processing for that tray. In this scenario, the server computing device 370 may automatically identify and analyze the timing of when the tray is received, automatically compare the identified time to a predetermined established time required to process that tray at the hospital, and automatically generate an alert for transmission to the various computing devices and medical devices of the hospital 100 when there is insufficient time to process the tray.

Additionally, the server computing device 370 may also utilize its knowledge (i.e., stored data sets for previous processes) of the flow of inventory through the SPD 122 and make further intelligent decisions. The decisions can be made according to programmed algorithms on the server computing device 370. For example, the surgeon may desire to know not just whether the loaner tray is delivered late, but whether the probability of that affecting his receiving a sterilized tray on time is reduced below 95%. In other words, the tray could be delivered 20 hours prior to the surgery, which is late according to the predetermined time, but the server computing device 370 can, in analyzing the flow of trays through the SPD 122, automatically determine or otherwise recognize that there is an opening in the use of the sterilization unit 110 such that the loaner tray has a high probability of still being properly sterilized and prepared in time for the surgery. Stated differently, the server computing device 370 can automatically determine, based on data (e.g., metadata) corresponding to other trays that have been processed by the system and its communicative connection to the various other devices in the SPD 122, that the late loaner tray may still be properly sterilized, or will likely be properly sterilized on time with a 95% probability. Thus, rather than generating a notification indicating that the loaner tray is late, the system may automatically make a decision not to generate the alert or notification.

Off Site Facility

In yet another aspect, a group of hospitals may outsource the sterile processing department to an off-site facility. This general arrangement is shown in FIG. 3C with configuration 350. A first hospital 352, a second hospital 354, a third hospital 356 and a fourth hospital 358 our each in communication with each other and can communicate trays amongst each other. One or more of the hospitals can include their own sterilization processing department (not shown). Of course any number of hospitals can coordinate with an off-site centralized sterilization processing department (not shown). In one aspect, the system disclosed herein manages the entire process of surgical instrument tray receipt, sterilization, and delivery between respective hospitals using the centralized SPD. The system interfaces with each respective hospital and can prioritize processing at the SPD or any SPD at any of the hospitals. The system will have access to one or more parameters that can influence the timing and priority of processing trays. The parameters can include the surgical schedule for each hospital, data about individual patients, data about the type of surgery, data about the manufacturer of instruments in an instrument tray, which hospital the tray is scheduled for, and so on. In this scenario, the same kind of management can occur including messaging, individual focused assignments for processing a tray, and so forth, can apply. In this context, the added layer of multiple hospitals being involved is all managed by the disclosed system. When trays are communicated to the SPD, they will include a tray number, a tray type, facility information, and so forth. Any gap or issue with timing can be reported to the proper distribution group. Thus, if a surgical tray is placed in storage and is identified to be there for a longer period of time than is expected, the notice can go out to a nurse was expecting that tray who can then reach out and communicate an instruction to an employee to find that tray and ensure that is delivered to the respective hospital. Again, in one aspect, the notice transmitted through the system to a nurse can be particularly configured with selectable objects for processing that particular tray. The system coordinates the processing of all of the individual trays and can properly report out the status of each individual tray based on the respective hospitals such that each hospital can have real-time insight into the status of their trays, although their trays in reality are mixed in with multiple other hospital trays in the SPD that are being processed. The real-time information can include information in transit such that GPS coordinates can be provided. RFID tags can of course be used in this context as well in which RFID readers can be provided on equipment or locations within the SPD, vehicles that may be used to transmit trays from a respective hospital to and from the SPD, and so forth.

Any functionality disclosed herein in the context of a single hospital, can be adapted to the concept of multiple hospitals utilizing a single SPD. The additional layer of information would include without limitation, for individual trays, the indication of the particular hospital that they are associated with or individual staff who has performed functions associated with the respective tray. Accordingly, all movement detection, biological indicator detection, notices, communications, and so forth can equally apply to the multiple hospitals scenario.

Messaging Application

The present disclosure presents a number of different innovations in the area of surgical asset management. The first relates to messaging which can include the generation of real-time alerts, notifications, and messages provided by the server computing device 370 for automatic and real-time transmission to respective client devices 306. The real-time alerts and messaging can address the following problem. Typically, if a surgical processing department employee is preparing a tray of instruments for a particular surgery, and is missing an instrument, the SPD employee Will call up to the nurse and simply ask whether they think they're going to need this particular instrument and whether they know where they can find one. This is a manual and slow process. Often times, the surgical tray gets sent to the operating room without that instrument and the nurses may need to improvise and either clean and instrument as best they can or substitute the desired instrument for the missing instrument. The current infrastructure for managing instrument trays does not take into account the potential issues that can arise as described above. Simple systems that record particular instrument trays in inventory simply do not contemplate the various important issues that arise in the overall process.

The SPD employee, in such a scenario as described above, can utilize client device 306 to provide input indicating that a surgical instrument tray 109 is missing a particular instrument, which may be transmitted to the server computing device 370. Based on the input, the server computing device 370 can automatically generate and transmit an alert and/or notification to the client device 306, at which users interested in the status of the respective surgical instrument tray 109 may interact. For example, there may be a team of nurses, a doctor, and an administrator that's associated with that tray, or a scheduled surgery.

The type of message that is delivered can be configured also according to time. For example, if the notice is transmitted six hours before an operation, then the notice may only need to go to a head nurse or other staff member. This can be because there is sufficient time to remedy the situation before the doctor arrives to perform the surgery. However, if the notice is provided a half-hour before the surgery, then the group of recipients of the message may be expanded because the likelihood of an issue arising needs to come to the attention of the doctor. In this regard, a dynamically generated and structured notice can be prepared according to a number of factors. As noted above, the timing issue can cause a different distribution list, or a differently structured notice with differently structured options to be presented.

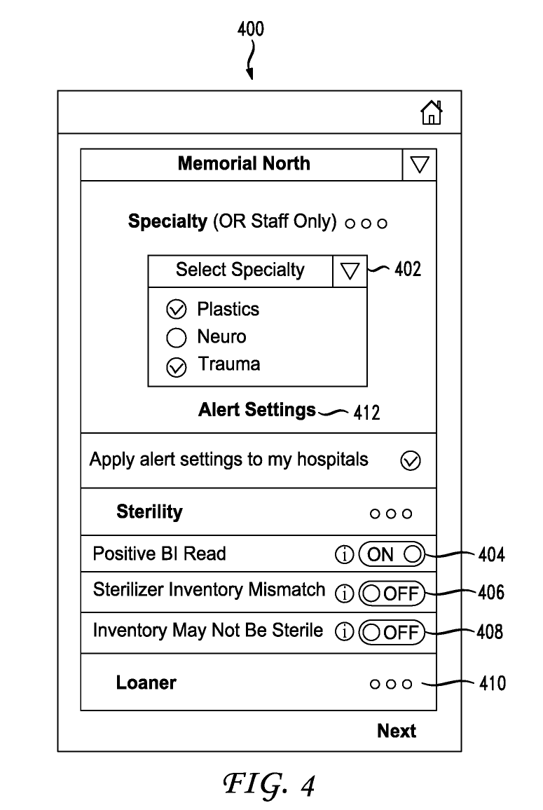

FIG. 4 illustrates an example graphical user-interface 400 that can be generated by the server computing device 370 and provided (e.g., served) for display at the client device 306 that enables a user to select a particular specialty for release for receiving notifications and alerts. Shown in feature 402 is the selection of plastics and trauma as areas of medical specialty. Of course any specialty could be selected by a user. The user can also select alert settings 412. For example, in the category of sterility, the user, as is shown in FIG. 4, wants notifications on a positive biological indicator (BI) read 404, but has turned off their alerts for sterilizer inventory mismatch 406 or if inventory may not be sterile 408. Other categories could include alerts for loaner instrument trays from other hospitals or entities 410.

The user may also be able to select and create additional people who will be added to the pool of individuals capable of receiving alerts at the client device 306. For example, if all orthopedic nurses are on an alert pool notification, a nurse can add in infection control people to the distribution list will automatically get added into the pool. The communication is expandable and extendable as necessary.

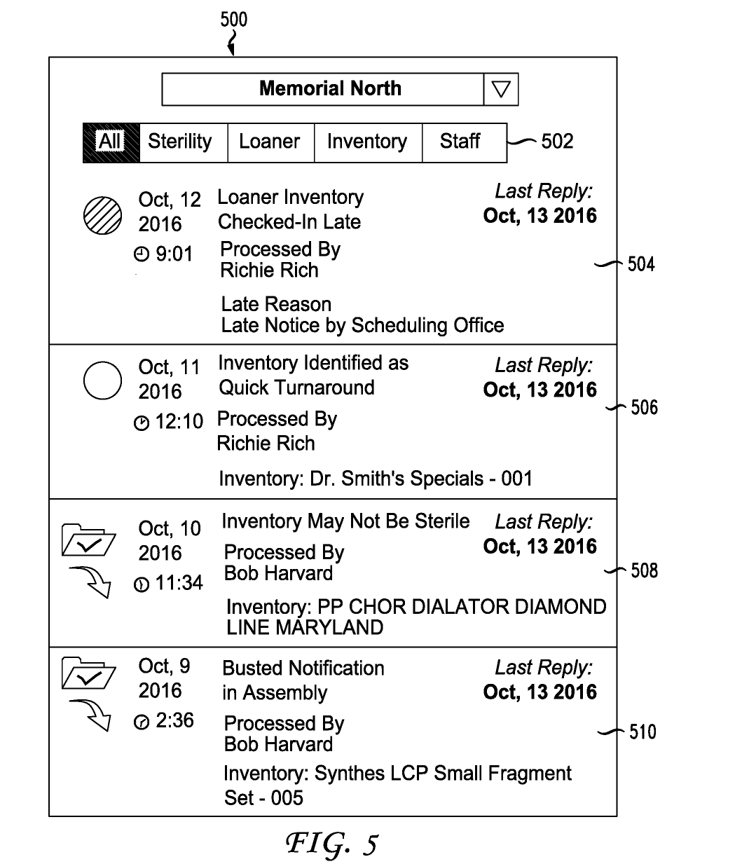

FIG. 5 illustrates example illustration of a graphical-user interface 500 (“interface 500”) that may be generated by the server computing device 370 and provided (e.g., served) for display at the client device 306. In the illustrated embodiment, the graphical-user interface 500 illustrates the various notifications that can be generated and transmitted. The interface 500 includes the option for a user to choose all notifications, justifications related to sterility, loaner sets, inventory, or staff 502. Example notifications include a loaner inventory check in late which is processed by a particular individual 504. A reason for the lateness of the loaner check-in is also provided. In some cases, a particular instrument tray may be tagged for a quick turnaround for accelerated sterilizations because it is needed in surgery. Such notification can also be provided as is shown in feature 506. In some cases, in the asset management process, there may be a question as to whether a particular instrument tray was sterilized or sterilized properly. A staff member, or an automated approach based on an analysis performed by an algorithm, can trigger a notification that inventory may not be sterile 508. This notification can go out to all of those in the particular department that need to receive that data. Feature 510 also illustrates another kind of notification for a “busted notification in assembly” which means that a particular set of instruments may be missing one or more instruments in the set, thus making it a “busted” set.

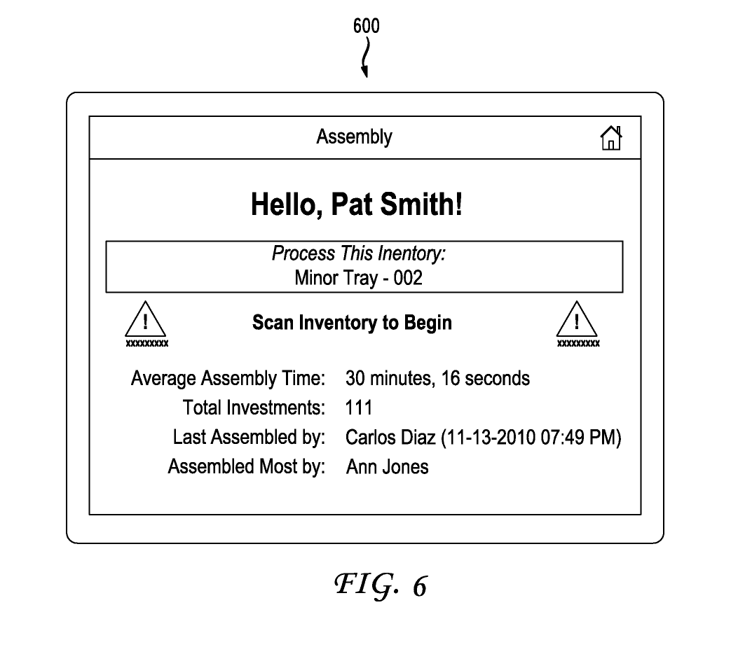

FIG. 5 illustrates one aspect of the various notifications that can be provided. In addition, the particular structure of the notification can be dynamic and selected or configured based on the particular functions that can be or should be performed with respect to that tray. For example, notification 510 is identified as being processed by Bob Harvard. The notification relates to a broken up set of instruments where perhaps one or more instruments is missing. The notification could be one that is provided to a head nurse a given period of time or within a threshold period of time prior to a scheduled surgery. The notification could include dynamically generated buttons which enables the nurse to provide easy instructions. One button could initiate the functionality of sending a “focus” message (See FIG. 6) two Bob Harvard to prepare the tray next and out of order for the surgery. Another object could be presented which, when interacted with by the staff member, could cause and alerts to go out to the entire team to find the missing instrument. The object could be preconfigured identify that instrument. For example, the object could indicate “Send Notice to all Staff to look for the Synthes LCP Small Fragment Set”. When the head nurse interacts with the object, that particular notice could be sent out to the proper distribution group. Accordingly, one aspect of this disclosure is analyzing an issue related to processing surgical instruments and generating a notification to one or more individuals with dynamically created functional objects which are presented for selection by the one or more individuals. Upon an individual interacting with the selected object, the functionality that was dynamically generated for that object is carried out.

Users may interact with the various components of the interface 500 to provide replies or instructions to the generated notices or indicate to the server computing device 370 that the notices and/or alerts should be automatically transmitted to another group of individuals, or be removed.

In one example, assume there are missing instruments in a particular type of tray, such as an orthopedic tray. All nurses signing up for orthopedic cases would receive the notice. The notice may indicate: “I'm working an orthopedic tray and a missing a long needle number 12”. Because the notice goes out to everybody (or could go out to a dynamically selected group of individuals), one of the nurses could identify that they have that particular instrument and the technician can come retrieve it and added to the tray for sterilization of preparation. The technician as well as supervisor can also see the communication flow. Infection control can also have access to the communication to see that the issue that was raised was resolved easily through the use of the system disclosed herein.

As noted above, additional dynamically generated functional objects can also be presented in the notification which can simplify the authorization an additional steps or functionality that would be initiated to remedy the situation.

Another example of the server computing device 370 in operation relates to confirming that a particular respective surgical tray is sterilized in the proper way. Depending on the set of surgical instruments, there are guidelines with respect to how to sterilize those instruments. Some instruments may need to be sterilized for a longer period of time than others. Some instruments may need to be sterilized to the higher temperature than others. For example, some instruments may have plastic materials that require a lower temperature for sterilization. Steam sterilization is often used to sterilize medical devices but other systems such as hydrogen peroxide gas plasma, parasitic acid immersion, ozone, have also been deployed. Often, a technician will prepare a number of different trays, each of which may have different sterilization requirements, and set a sterilization unit 110 with parameters that should match all of the various different requirements of the different trays in the sterilizer player. However, if there is a mismatch, the server computing device 370 can automatically detect such a mismatch. Using the RFID tags, and the data contained within such tags, the server computing device 370 can automatically identify if a particular respective surgical instrument tray is being sterilized in a sterilization unit 110 with parameters that do not match the instruments. For example, the sterilization unit 110 may be set to steam sterilize at 270° for 10 minutes. One of the 20 trays in the sterilization unit 110 may require 15 minutes of sterilization. In another scenario, many of the instruments may be over cooked in the sense that they either have a temperature that is too high, or are in the sterilizer too long. This can damage or weaken the instruments over time. The server computing device 370 automatically tracks the sterilization parameters for each individual tray and can automatically generate an alert and/or notification that identifies the mismatch based on the tracked parameter data. The server computing device 370 can generate and indication that a particular tray should be removed from the load, but the employee 126 may sterilize the particular tray in the load anyway. After sterilization, the employee 126 may put the tray into inventory and store it theoretically ready for surgery. The tray may be barcoded or tracked with an RFID and identified as being in storage after sterilization. In this scenario, however, where a question may arise because the processing of the respective tray of instruments has been out of a normal expected procedure, an alert can be generated to the appropriate team that this tray may not be sterile. The server computing device 370 determines that it may not be sterile because it doesn't have an electronic record of ever being sterilized, because the server computing device 370 removed an electronic record of the tray from the load.

The notifications can also be generated and transmitted in real-time. For example, the mismatch can be identified prior to the technician 126 entering the surgical instrument tray into the sterilization unit 110. The additional technology deployed as disclosed herein, which can include an RFID reader 334 which can detect which particular trays are being inserted into the sterilization unit 110, can coordinate the needs of each individual tray, with the parameters established for that sterilization session, and initiate an alert to the staff member 126 that there is a mismatch with that particular batch. The alert could be presented on a graphical display associated with the sterilization unit 110 or can be mitigated through the system to a mobile device associated with the staff member 126. The notification or alert can also go out and an orthopedic nurse can respond via the application (e.g., deployed on the client device 306) with the comment that indicates that they are trying to put that tray into the wrong load. The nurse may get a later message as noted above which indicates that the tray may not be sterile.

In this scenario, the timing of the alerts in real-time or near real-time is valuable in that the nurse may have a patient that is just being put under anesthesia. If there is an issue with the sterilization of the tray having instruments to be used for that surgery, the nurse may require that the tray be re-sterilized with the proper parameters immediately so that the problem is fixed. Supervisors are included in the communication between the individual so that they can reply back with comments like “wonderful job.” The goal is to solve issues before the instrument tray is on the back table and a surgical room ready to be used but perhaps not sterile. The reply back from a supervisor can be indicated via interaction with a selectable object which is dynamically presented to a supervisor interface after the problem is resolved. Thus, in one aspect, the various types of notifications or messages that can be communicated to particular individuals through the cycle of identifying an issue with respect to processing a surgical instrument tray, to notifications and assignments of specific tasks in order to remedy the situation, and finally notifications which can indicate a successful resolution, can each be dynamically generated and tailored for the particular stage of the process. For example, after several notices have gone out in order to attempt to remedy the situation, if the particular tray has not yet been sterilized as instructed, the notification to a senior-level supervisor can include an object selected which can indicate their instruction to immediately stop all other sterilization processes and process that tray. The other hand, if the situation was remedied efficiently via individuals carrying out the instructions as provided through the notifications and interactions disclosed herein, a selectable object by a supervisor can include a praise statement such as “great job to the team.”

Utilizing the tray tracking capability, and the coordination and communication functions of the server computing device 370 included in the computing network/environment 300 and the individual applications loaded onto user's mobile devices (e.g., at the client device 306), an important component of the present disclosure is the real-time communication that is now possible and that represents an improved functionality that was not previously possible. Previously, without the coordinated functions of the server computing device 370 and overall system disclosed herein, there was always a gap in timing or a gap in information that was inherent in the process.

Thus, using the various computing systems disclosed herein, notifications with respect to missing instruments, dynamically generated interfaces, mismatches and sterilization procedures, high-priority cases, and so forth can be automatically generated and immediately communicated to various computing devices and/or medical devices included in the hospital 100, thereby enabling staff to immediately interact with such devices such that issues can be fixed more quickly and efficiently such that a sterile, complete and accurate surgical instrument tray can be delivered to the back table for surgery at the proper time.

In another aspect, there are many surgical trays that are processed every day. If there is a particular surgical tray that needs a quick turnaround for returning back to another surgery, this can often be difficult. Currently, there is a list of processing priorities that can be based on a surgical schedule which on the surface sounds workable. The reality is, however, that employees 126 often are going to simply sterilize trays in the order that they desire. The priorities are difficult to enforce simply because of the volume of surgical trays that need to be processed. Thus, a focus window can be established within the application which can take the surgical schedule into account as well as individual instructions from the appropriate staff person. FIG. 6 illustrates an example graphical user-interface 600 generated by the server computing device 370 that represents a focus window directed to a particular tray and to a particular person. The focus window will be served or otherwise provided to on the client device of Pat Smith in this example with instructions to process minor tray-002. The focus window instructs Pat Smith to scan the inventory to begin the process. Estimated times are provided for how long it would likely take to assemble the tray, how many instruments on the tray, last reassembled by a particular individual as well as information about the individual who has most of the time assembled the tray.

In this regard, the server computing device 370 automatically tracks individuals and their experiences with trays such that if necessary, the server computing device 370 could automatically perform an analysis of technicians within the SPD 122 and coordinate accelerated processing with one or two or more individuals in order to get the tray done as quickly as possible. For example, if Carlos Diaz was the person who last assembled the tray, and Pat Smith has never assembled the tray but is perhaps established in the server computing device 370 (e.g., stored in a database mapping technician data to tray data) as a person who should handle priority cases, the notice can go to Pat Smith as well as the Carlos Diaz who might have the instruction to immediately go help or monitor the assembly of the tray as they were the last one to do it. Or, the server computing device 370 can automatically transmit a notification including an instruction or the assignment to assemble the tray to Amy Jensen who was the one who is assembled at most and thus might be the most efficient. The decision on which person or group of people to send the assignment to may also depend on other factors, such as, the seriousness of the surgery (is it a knee replacement or triple bypass surgery), characteristics of the patient such as their age, health condition, emotional condition, and so forth. Any number of factors could be analyzed by the server computing device 370 to determine how urgent the need is, a particular threshold of expected efficiency that may be needed, and so forth. Thus, in a most urgent scenario, where the highest trust in the process is needed, the primary assignment can be sent to the most experienced and efficient person in handling that particular tray of instruments with additional messages to the last person that assembled the tray and others as well. Previous timing of individuals who assembled the tray could also be tracked by virtue of data input, barcode tracking, RFID tracking, and so forth. Thus, an algorithm that determines which person or group of people to give an assignment to assemble particular tray can include their historical experience and proficiency, accuracy, training, skill set, and so forth associated with the experience.

In addition, given the scenario as set forth in FIG. 3C, where individuals may move from hospital to hospital, a centralized database can be utilized to maintain the historical tray experience for individuals as they moved to a new hospital. To the extent that essential management system can utilize Global ID information for respective trays, the following scenario could occur. Assume that John Smith works at a first hospital and is the most experienced person at organizing the minor tray-002. Normally, if John Smith moves to a second hospital, that historical information about his experience level with particular trays would be lost. Utilizing a centralized approach, with the ability to globally identify instrument trays, as John Smith begins work at the second hospital, a focus screen like is shown in FIG. 6 could still indicate that John Smith was the last one to assemble the minor tray-002, even if it was at a previous hospital. If there is consistency in identifying trays across hospitals, then the accuracy and usefulness of the information can transition with individuals from one hospital to the next utilizing the system disclosed herein.

Furthermore, the application which provides the assignment to process a particular inventory may include specific assignments to be the lead on assembling a tray to a first person, and a specific assignment of the second person to be the assistant to the lead. Thus, Pat Smith may receive a notification or a focus window that indicates that she is the primary lead on preparing this tray immediately and that Amy Jensen has been asked to assist in assembling the tray.

Inasmuch as the server computing device 370 also tracks all of the inventory and knows, for example, what inventory is currently being sterilized, what inventory is packaged and ready to go to surgical rooms, and so forth in a rush scenario, the server computing device 370 not only could provide notifications to particular individuals to process an inventory tray, but could also send other notices to other individuals downstream to render the process more efficient. For example, John Smith might be managing the sterilization unit 110 that day and the current batch may be scheduled to be finished in 10 minutes. If Pat receives a notice as shown in FIG. 6 to begin processing a tray immediately, John Smith may get a notice that a particular tray with particular sterilization requirements is going to be ready in approximately 20 minutes for sterilization. This can prompt John to more quickly remove the batch of trays that are being completed in the sterilization unit 110 and prepare the parameters of the sterilization unit 110 for the rush tray of instruments. In other words, the server computing device 370 can send one assignment to a first stage of the process of managing trays of surgical instruments and send a second assignment to a later stage in the process in order to prompt and coordinate the individual workers for the task at hand.

Given the notifications that have been provided to the server computing device 370, if the employee simply does not quickly turn the instrument tray around as instructed, it will be documented in reportable that the employee bypassed the particular message and alert can be also sent to a supervisor or manager. For example, the notice to prepare particular tray cannot only go to the employee 126 but also to a nurse 122 who can recognize that the requested tray has not been barcode scanned, RFID detected, or placed into the sterilization unit 110. The system enables the nurse to provide immediate feedback such as, “John, you cannot skip this, please proceed to process it now.” In one aspect, the window in FIG. 6 can be generated by a staff member but in another aspect is automatically generated based on one or more factors. The generation by the staff member can indicate that the staff member types in a particular message, or selects a selectable object which carries of the functionality programmed into the object. As noted above, the one or more factors can include the surgical schedule, historical timing of preparing the particular instrument tray for the surgery, characteristics of the patient, characteristics and historical needs of the nursing staff or surgical staff, the current state of inventory and surgical instrument management, and so forth. For example, the system may know that there is nothing scheduled to be sterilized over the next six hours and that under normal processing, the requested tray should easily be completed in time for the surgery the next morning. In one aspect, a particular surgical instrument tray is matched to an employee.

In some cases, the most experienced employees recognize which tasks are the most difficult and complicated in terms of processing surgical instruments. But human nature, they may push those more difficult projects onto newer less senior employees. Thus, often what happens, is the most seasoned people are working on the least complicated and most simple instrument trays. The least competent people, or the newest people in the department, are often assigned to work on the most complicated trays. This reality can lead to more problems and surgery. The novel features disclosed herein prevent this issue from arising because the system will make individual employees accountable particularly when they're assigned to process a particular tray. A more senior seasoned worker will not be able to simply pass of the more difficult tasks to less competent workers. Thus, in one scenario, if the employee is new and has never prepared that particular instrument tray, an alert may be automatically generated which instructs them to prepare the tray, and optionally can include an alert to a supervisor or another employee who has had experience in preparing that tray. In another scenario, if the employee associated with that instrument tray is very experienced, then no alert may be needed. Thus, this concept involves using the server computing device 370 to perform new functions with respect to how to provide directives to specific employees to prepare specific surgical instrument trays. Furthermore, the directive can be more complicated in creating a team for managing the preparation of the instrument tray as well as directives to others in the chain of operations needed to prepare pair the instrument tray completely for surgery.

Another aspect of the present disclosure relates to gap management. In some scenarios, a tray may finish its sterilization process, get taken out of the sterilization unit 110, set on a shelf and forgotten. With the RFID technology, the system can track each individual tray such that the system can have expectations on where the tray is at each stage. For example, it may be expected that the tray will begin to be moved up to the surgical room within one hour after being removed from the sterilization unit 110. However, if the tray ends up sitting on a shelf for 1.5 hours, and the staff member forgets about it, or it actually gets misplaced, then the server computing device 370 will automatically recognize (e.g., via metadata and tracked data obtained the system) that the tray, according to the RFID 328 and the reader 336, has not left the SPD 122. In that case, an alerts can go out to the proper individuals such that a staff person 126 can identify the tray and send it up to surgery. Similarly, an issue could arise where a tray is sitting and waiting to be sterilized, and it is expected that it will be in a next batch of trays for sterilization, but it is not. The RFID reader 334 can identify that the tray did not make it into the batch for cleaning. This causes a gap in the process that typically would take a long time to identify. Again, the staff 126 is typically processing hundreds of trays a day and will not capture every gap of time.

One example of minimizing the gap is that the system, knowing the inventory status of processing, and knowing that a particular tray is needed for surgery but that it has been sitting too long after coming out of the steam sterilization unit 110, can also identify that Pat 126 has walked up to device 326 which has an RFID detector 330. Pat has an RFID tag as well 332. The server computing device 370, in receiving or otherwise obtaining all of these particular pieces of information, can initiate a communication via the computer 326 that says “Pat, I know it's you, I know the most important tray. Please proceed to process this tray and get up to surgery by 11 AM.” Thus, the system retrieves information about a specific surgical instrument tray, receives information about a location of an individual that is associated with the surgical tray, and presents a notification on a device associated with the individual, with instructions to process a particular tray. The notifications can be automatically generated, or triggered by a supervisor interacting with an object, can be dynamically generated to include objects which Pat can interact with to confirm the message, confirm that the task is complete, send questions or other notifications, and so forth.

In another aspect, with the system in place as is shown in FIG. 3A, coordination between surgical instrument management procedures and other systems within a hospital can be achieved. For example, assume a patient arrives in the ER 102 with a bullet wound. That patient is immediately going to go to surgery. Currently, there is no communication between the ER and what tools may be needed by the surgeon for this particular surgery. The present solution provides a number of different mechanisms and/or processes that automatically change parameters within the server computing device 370 in order to modify how instrument trays are processed and delivered. For example, information associated with the gunshot wound patient can be entered into the system any different number of ways. A nurse can use the application on his or her device 306 to provide parameter information highlighting the particular needs. An entry in an electronic health record maintained at an electronic health record computing system may be accessed by the system that indicates a gunshot wound to the chest, which can automatically initiate a change in a particular parameter. For example, assume that the instrument tray that would be needed for performing surgery on a gunshot wound patient is in processing in the SPD 122. Assume that a previously existing parameter associated with a timing of how long it is before the tray is needed is one hour. With the entry of the new information to the server computing device 370, that gap time can be modified to five minutes. An alert can be issued, such as the focus screen 600 shown in FIG. 6, to inform a particular user that there is now a new priority of which tray to process and to immediately process that tray so that an accurate and sterile instrument tray can be delivered to the ER.

As noted, the initiation of the information into the server computing device 370 which can cause a change in the timing parameter of the surgical instrument management system can occur in any number of different ways. For example, an interface can be provided to an ambulance or paramedics who can have mobile devices with applications that interface with the system disclosed herein. A button in the ER, through the application disclosed herein in which is provided on mobile devices, or in any location can be provided. Preferably, the interface between the electronic health records and the surgical instrument management system is a potential location which will initiate the change in parameters.

It is noted that any one or more parameters in the instrument management system can be adjusted. For example, parameters associated with sterilization, associated with an order or priority of processing, a timing associated with any stage in the process, which process is assigned to a particular tray, or what team is to be assembled to oversee the configuration, sterilization, and delivery of a particular tray. For example, if a particular tray is assigned to a relatively new employee, and the information received in the system indicates that an emergency is occurring and that tray needs to be accurate and delivered immediately, the system not only may change a timing parameter associated with the tray but may assign a senior staff member as well as the last person that assembled the tray to prepare it for surgery.

The trigger that modifies parameters within the surgical instrument management system could also be based on the individual who was arriving in the hospital. For example, if a high government official or the president of the hospital, or some particular individual is identified, and potentially parameters may change based on that individual. For example, different instruments may need to be provided for somebody who is quite young relative to somebody who is quite old. A weight of the patient may be an indicator of a different set of instruments may be preferable over the set that is already in the operating room. Thus, there are any number of different scenarios in which information associated with the patient can be used by the system to automatically modify parameters associated with processing a surgical instrument tray and any number of these parameters, including one or more parameters in combination, could be applicable to make modifications to existing or default parameters.

Sterilization Confirmation

Another aspect of this disclosure relates to using the server computing device 370 to automatically manage issues with sterilization of trays. The sterilization unit 110 will typically process a batch of trays. Not every tray of instruments will have the exact same requirements for sterilization. However, typically, the technician will put in a batch of trays that will at least have parameters that can be met by a common denominator of settings on the sterilization unit 110. For example, a set of 20 trays may each require at least 270° of temperature, four minutes of exposure, and 40 minutes of drying time. The technician 126 will scan the barcode of all of the 20 trays that are going into the batch. In one aspect, each tray will have an RFID which can be used to identify through RFID reader 334 which trays go into the sterilization unit 110. Typically, after the sterilization process, the sterilization unit 110 prints out a confirmation of everything that was in the batch. The receipt from the sterilization unit 110 can report that the batch met the requirements with respect to exposure time, temperature, chamber pressure and so forth. However, in some circumstances, that report can identify that a load failed. The sterilization unit 110 can report that failure electronically as well as through the printout. However, errors can occur where the technician 126 may simply take the tray out and move it along the assembly line to be taken up to the ER 122. With the communication from the sterilization unit 110 to the system, the system can identify each tray that was in the batch and report that it may not be sterile. Currently there is no recall process for such a scenario. There may be a small printout or a change in a color of a tape used to hold down linens over the tray due to the heat in the sterilizer, but there is no system or mechanism to recall or track the potential nonsterile tray. What can happen is a nonsterile or questionably clean tray can reach the back table in the operating room and a nurse can then potentially identify, once the patient has been anesthetized, that the instrument tray may not be sterile. Then, they must send the set down to the SPD 122 for a quick sterilization, all of which is damaging to patient care.

The solution according to the principles disclosed herein is that the disclosed system tracks the processing of each individual tray in real time and provides the necessary notices and instructions to prevent the above scenario from happening. In one aspect, a communication can go out to one or more individuals on a distribution list associated with a nonsterile tray. For example, a nurse was expecting that tray for a surgery in the afternoon can see instantly and in real-time information that the tray has come out of the sterilization unit 110 without a confirmation that the sterilization process was completed. In another aspect, the server computing device 370 could generate an interface in which the proper individual will receive a notice or an alert about the tray and has to manually engage with or interact with an object on an interface to release the tray notwithstanding the warning.

Thus, if a tray comes out of the sterilization unit 110 with a questionable state of sterilization, the server computing device 370 can quarantine the tray, automatically provide notices through an interface regarding the potential issues with the tray, and potentially present a question and answer session which enables the proper person to either take responsibility for the tray and release it, or provide instructions to the proper person to re-sterilize the tray, or perform some other action. For example, the sterilization unit 110 may not have properly done its job according to the requirements programmed in the system. Perhaps the temperature wasn't quite high enough, or that heat was not applied as long as it should have been. However, as the system typically is programmed for the lowest common denominator of trays within a batch, the proper person could review those requirements and note that for the particular tray, the requirements were actually met and release this tray so they can proceed to the operating room.

In this scenario, a number of different parameters and triggers can also be combined when determining what action to take. For example, depending on the timing of when the tray is needed, and the nature of the surgery, a new person may be assigned to be responsible for the tray who is a more senior or more experienced person than was originally assigned. Gap times in the system for when things the need to be processed may be shrunk or extended. For example, in one scenario, if the surgical tray is not needed immediately, and the knowledge of the overall inventory status of the system indicates that there is an opening in the afternoon for sterilizer, the system can adjust the parameter such that the instrument tray can be scheduled to use the sterilization unit 110 at an open time with sterilization parameters set just for that tray. One or more of the following parameters can be utilized to make decisions in the system for a potentially nonsterile tray: the type of surgery the tray is associated with, data about the particular patient, data of the overall scheduling of operating rooms, data associated with the overall inventory flow of other instrument trays in the hospital, data of loaner instrument trays and status, characteristics of the doctor performing the surgery, manual information input by a user, qualifications and characteristics of individuals in the SPD 122 that are handling the instrument trays, and so forth.

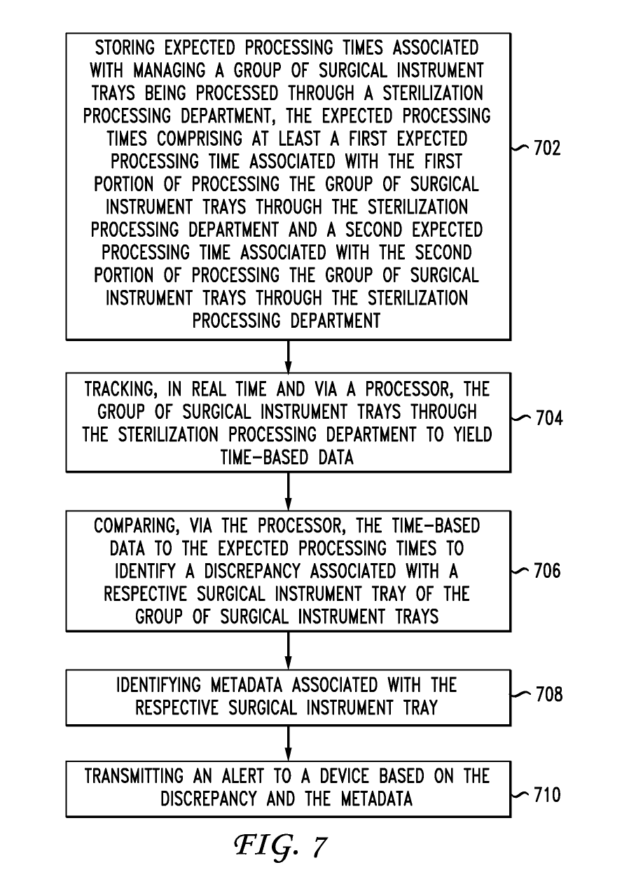

FIG. 7 illustrates an example method implemented through a messaging application according to this disclosure. The method includes storing expected processing times associated with managing a group of surgical instrument trays being processed through a sterilization processing department, the expected processing times including at least a first expected processing time associated with the first portion of processing the group of surgical instrument trays through the sterilization processing department and a second expected processing time associated with the second portion of processing the group of surgical instrument trays through the sterilization processing department (702), tracking, in real-time using the server computing device 370, the group of surgical instrument trays through the sterilization processing department to yield time-based data (704), comparing, using the server computing device 370, the time-based data to the expected processing times to identify a discrepancy associated with a respective surgical instrument tray of the group of surgical instrument trays (706), identifying metadata associated with the respective surgical instrument tray (708) and transmitting an alert to a device based on the discrepancy and the metadata (710). As noted above, the alert that is transmitted to a device can also be dynamically generated to include one or more selectable objects which, when interacted with by a staff member, can implement specific functions particular to needed or desirable processing for the particular instrument tray.

The method can further include receiving an instruction from a user of the device. Based on the instruction, the method can include identifying a responsible person for the respective surgical instrument tray and transmitting a message to a second device associated with the responsible person that instructs the responsible person to prioritize a processing of the respective surgical instrument tray. Instruction from the user of the device can involve user interaction with a selectable object which identifies the function to be carried out.

The method can include the tracking in real-time involving receiving tracking data for each surgical instrument tray of the group of surgical instrument trays based on a radio frequency identification tag physically associated with each surgical instrument tray. The system stores expected time gaps for the processing of each respective tray and compares those expected time gaps to actual time frames identified through tracking each surgical instrument tray through the process. An example discrepancy can include a late check-in of a loaner surgical instrument tray from another hospital or another entity. The discrepancy can also include a delay in a particular parameter associated with expected processing times. In one aspect, the particular parameter can include one or more of: a late check-in time for a loaner surgical instrument tray, a storage time, a sterile processing time, a time associated with respective surgical tray being at a particular location. Other possible messaging alerts can be provided as follows: (1) Loaner check-in in which an alert is sent to users notifying them of any loaner inventory checked in, (2) Loaner checkout in which an alert is sent to users notifying them of any loaner inventory checked out, (3) Inventory Not Sterile (Send or Case/Cart Module) in which an alert is sent to users when inventory without a sterilization record was sent to a location, (4) Quick Turnaround Inventory Marked in Decontamination/Assembly in which an alert is sent to users when an inventory item was flagged for Quick Turnaround, (5) Sterilizer Mismatch Notification in which an alert is sent to users when an inventory item was scanned into a sterilization load with unapproved parameters, (6) Incomplete Tray (by Specialty) in which an alert is sent to users when a tray was assembled incomplete, (7) Inventory Needs Repair (Triggered by Repair Scheduler) in which an alert is sent to users when an inventory item has reached a designated repair threshold, (8) 60-Second (Busted) Assembly Warning, (9) A Priority Window Override notice, and (10) Tray Marked Quick Turnaround Scanned into Sterilizer in which an alert is sent to update on any inventory previously identified Quick Turnaround inventory that has been scanned to a sterilization load.

Other alerts can also be provided or otherwise generated by the system as well. For example, the following are additional alerts which can be triggered based on the respective information described : (1) Incomplete Case/Cart Processed can cause and alert to be sent to users if a Case/Cart was setup completed with necessary inventory missing, (2) Comment Received by O.R. Comment Center in which an alert is sent to users when Operating Room staff have added a comment on a specific inventory set or instrument, (3) Request Loaner To Representative (requires manual typing—feature) in which a user requests certain trays based on specific surgeon and specific case which sends an alert to a loaner representative, (4) Scope Needs Re-Processing in which, based on a previously set frequency, an alert is sent to users notifying that a scope requires re-processing, (5) Lost Instrument in which the system sends an alert to users when instruments sent to a surgical procedure were not returned to sterile processing, (6) Priority tray scanned in Decontamination in which the system sends alerts two users users about a tray previously identified as priority has arrived in Decontamination (is now available for sterile processing to handle), (7) Processing flow deviation in which an alert is sent to users when inventory has skips a process in a defined flow, (8) No Surgical Scheduling Interface file received in which an alert is sent to users when no surgical scheduling interface file was received by the processor, (9) No Sterilizer Interface received in which an alert is sent to users when no sterilizer interface file was received by the processor, (10) Peel pack created in Assembly in which an alert is sent to users when new peel pack items are added in the database at the time of assembly, (11) New loaner created in loaner in which an alert is sent to users when new loaner sets are added in the database at the time of Loaner Check-In, (12) Case/Cart completed after case start time in which an alert is sent when a case cart is completely setup after the designated case start time, (13) Immediate Use Steam Sterilization Load Alert in which an alert is sent to users when any immediate use steam sterilization load is processed, (14) Bad Labeling Alert in which an alerts is sent to users when a new label for inventory is printed in the assembly module, (15) Inventory Processed 2× or More in a Day (Tray Shortage) in which an alert is sent to users when inventory is processed 2 or more times in a day to identify a potential inventory shortage, (16) Inventory added to “Waiting for Repair” List in which an alert is sent to users when an inventory item is removed for repair during Assembly process, (17) Sterilizer Load Weight Exceeded in which an alert is sent to users when instrument trays in a sterilization load exceed a designated weight amount, (18) Inventory not processed in last “x” months in which an alert is sent to users when inventory has not been processed after a designated amount of time to monitor database accuracy or find inventory and better utilize instruments within it, (19) Critical Instrument missing in assembly in which an alert is sent to users when an instrument identified as critical has been marked missing during the assembly process, (20) Every x (configurable) tray processed in assembly in which the system sends an alert to users on a frequency based on a configurable number of trays assembled, (21) Every x (configurable) tray processed in Sterilizer in which the system sends an alert to users on a frequency based on a configurable number of trays sterilized, and/or (22) 7:00 AM Snapshot Summary: Previous day summary (until 6:59 am) in which the system sends an alert to users on a set frequency summarizing department productivity. Any one or more or combinations of these parameters can be utilized to provide customized alerts.

Biological Indicator Alerts

In another scenario, one mechanism of confirming that a particular batch of instrument trays was properly sterilized is through the use of biological indicators. A biological indicator is a file or entity that is placed within a batch of trays that is sterilized. The file is tested for a period of time after the conclusion of the sterilization of the batch to determine whether bacteria has grown in the file. If so, then the bacteria was not killed in the sterilization process and there is a question about the sterility of the trays in that batch. The period of time needed to confirm a sterile batch can be a short amount of time or sometimes 24 hours. A problem can occur where the biological indicator file can be put into an incubator after the sterilization process and some hours later, when bacteria grows, the batch of instrument trays that were thought to be sterile are confirmed to not be sterile. Such instruments by that time may have already been used on a patient. Some trays may be in the process of being moved to an ER 102 or simply at some location in the pipeline. One aspect of this disclosure is, based upon a positive reaction to the evaluation of the biological indicator, includes the system sending an alert to each individual in the distribution group to notify everyone of the issue with each instrument tray. In this regard, a listing of each tray in this circumstance can be provided to an employee who can track down all of the inventory easily to take each tray out of circulation.

In circumstances where that instrument tray may have been used on a patient, that patient can be identified so that appropriate action can be taken to minimize any potential effect of non-sterile instruments having been used.

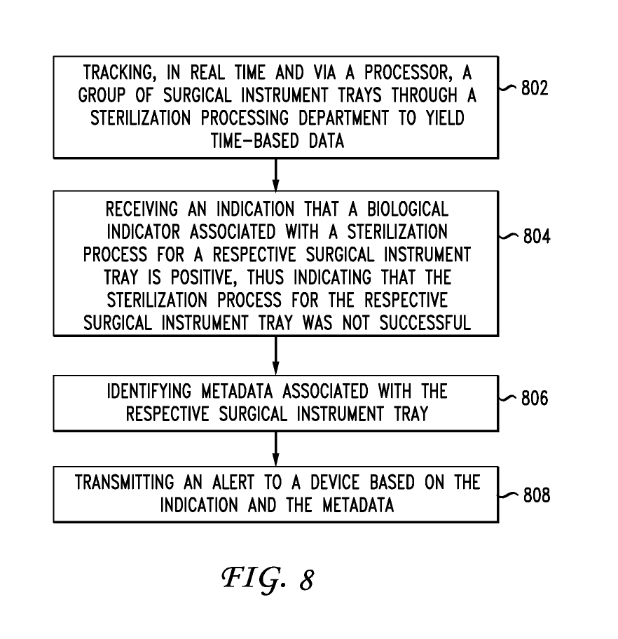

FIG. 8 illustrates a method embodiment for the use of the biological indicator. The example method includes tracking, in real-time using the server computing device 370, a group of surgical instrument trays through a sterilization processing department to yield time-based data (802), receiving an indication that a biological indicator associated with a sterilization process for a respective surgical instrument tray is positive, thus indicating that the sterilization process for the respective surgical instrument tray was not successful (804), identifying metadata associated with the respective surgical instrument tray (806) and transmitting an alert to a device based on the indication and the metadata (808).

The method can further include tracking each surgical instrument tray in a batch associated with the sterilization process, establishing a quarantine process for each surgical instrument tray in the batch and transmitting a notice associated with each surgical instrument tray in the batch.

In another aspect, the method includes, based on tracking each surgical instrument tray in the batch, determining whether a patient received medical care using one of the surgical instrument trays in the batch. The alert that is provided can be dynamically generated to include selectable objects based on the data or metadata associated with the alert. For example, if a first patient and a second patient were treated with instruments that were identified as not confirmed to be sterile, then a notification to an administrator can include a selectable option to provide the proper notifications to the first patient and the second patient, as well as the staff, to initiate procedures to minimize infection or other potential issues. Another selectable object can include a quarantine order for trays within the batch that have not yet been used for surgery.

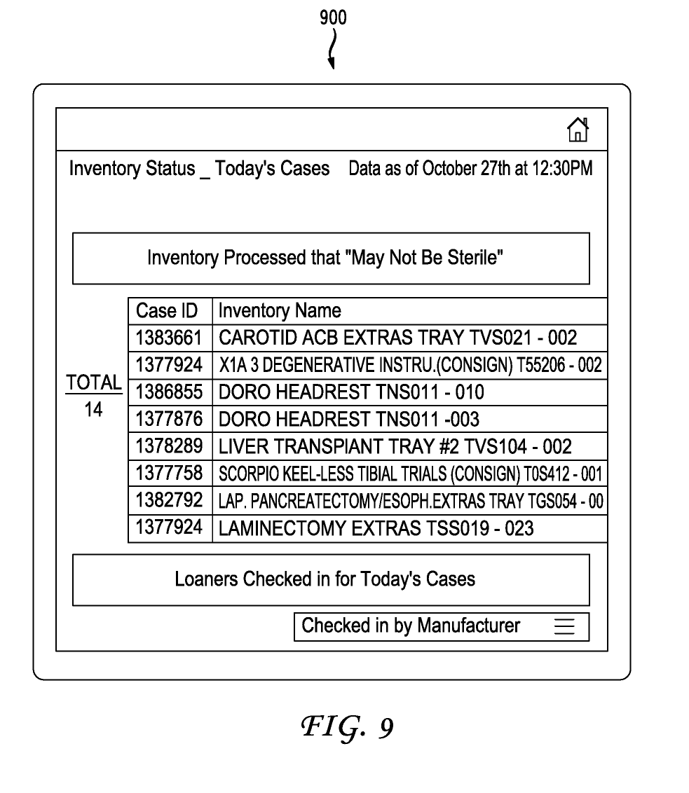

Next the disclosure provides a discussion of various dashboard features which can be generated by the server computing device 370 and provided through an interface (e.g., at the client device 306) to one or more users. The interface can be a browser or an application or any other client technology which configures the graphical features disclosed herein. The information provided draws upon the knowledge of the processing of surgical instrument trays as disclosed herein and can present summaries and analysis results from the system to individual users. FIG. 9 illustrates an example graphical-user interface 900 that may be generated by the server computing device 370 showing the status of inventory for a particular day in a. This particular interface shows a total number of inventory trays process that may not be sterile. Interface includes a case ID and an inventory name. Inventory may end up on this list by virtue of being detected in some of the scenario set forth herein. For example, an indication that the sterilization unit 110 failed to perform according to the expected parameters, or indication that a biological indicator test failed, and so forth.

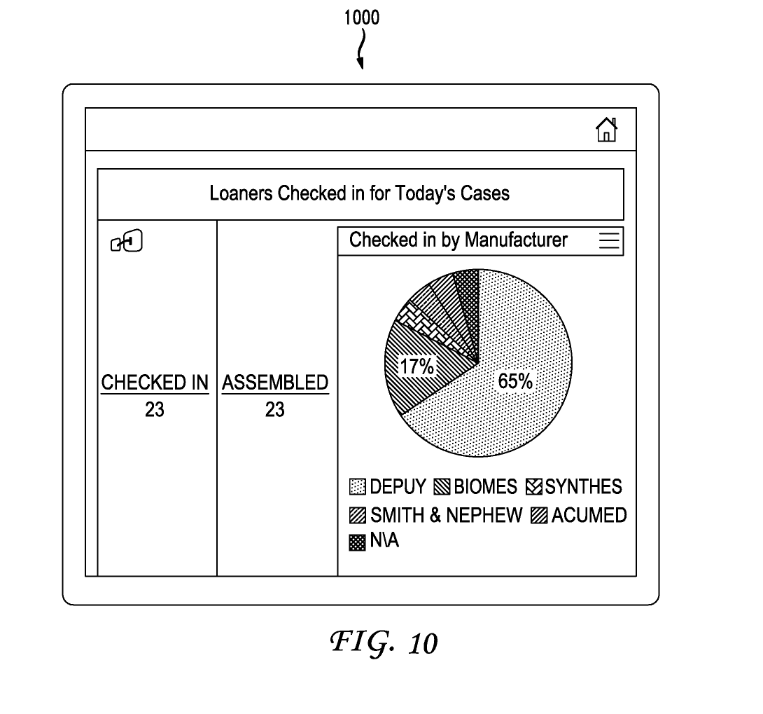

FIG. 10 illustrates an example graphical-user interface 1000 that may be generated by the server computing device 370, which identifies loaner trays that are checked in for a particular day's cases. In some cases, trays may be loaned from other hospitals or clinics and the inventory tracking system can identify particularly loaner systems are checked in, assembled, and present a graphical feature that indicates which loaners are checked in according to manufacturer.

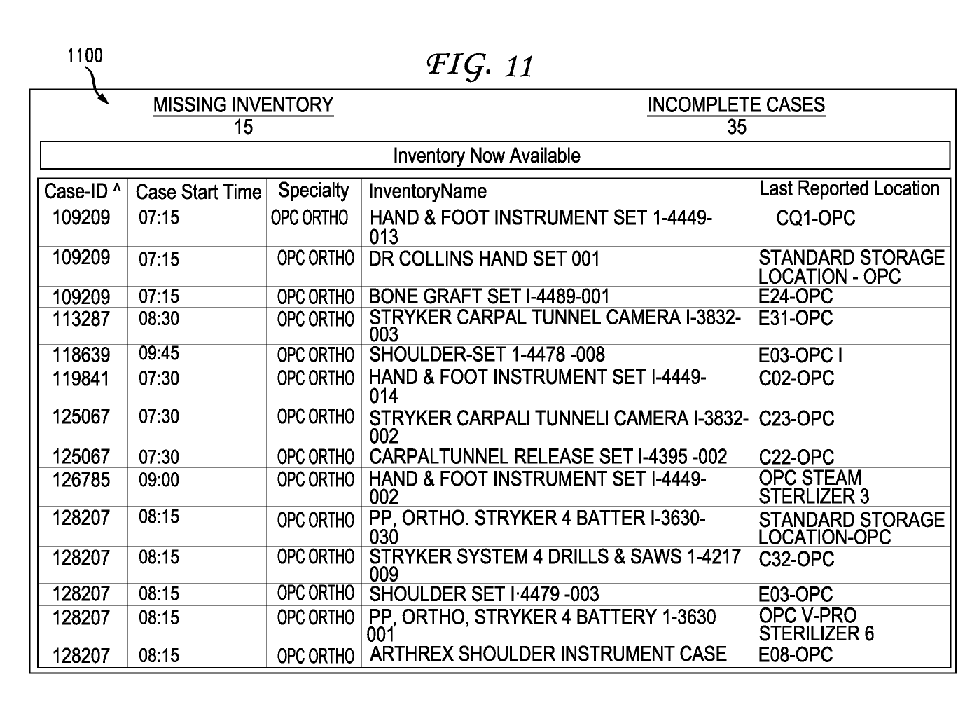

FIG. 11 illustrates an example graphical-user interface 1100 that may be generated by the server computing device 370, referred to as a dashboard view of interface 1100 that indicates both missing inventory, incomplete cases, in inventory now available. Inasmuch as the system tracks in real-time the location of individual trays, the information about inventory available can include a case ID, a case start time, a listing of a specialty, and inventory name, as well as a last reported location of that instrument tray.

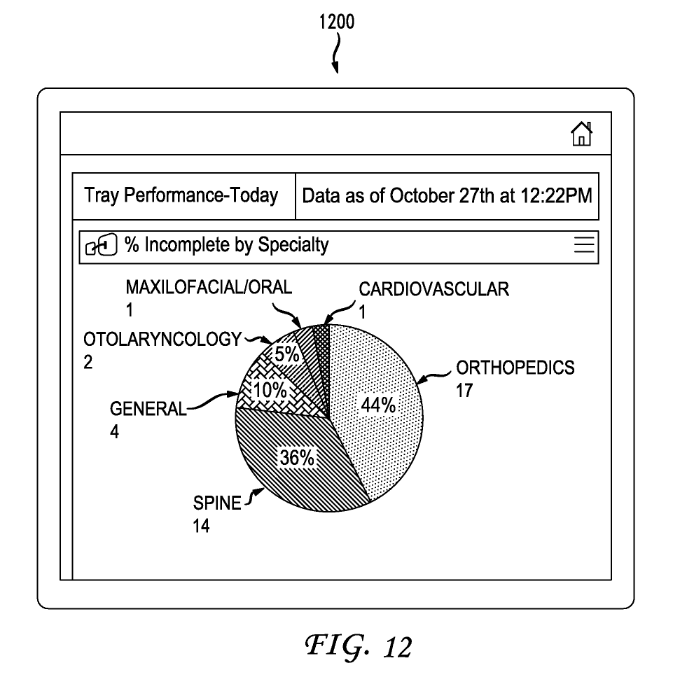

FIG. 12 illustrates an example graphical-user interface 1200 that may be generated by the server computing device 370, of tray performance for a particular day. As is shown, a graphical presentation 1200 can include a distribution by specialty, such as orthopedics or spine, and which trays were incomplete in each particular specialty. Thus, viewing the chart shown in the figure, most of the incomplete trays were identified as being part of orthopedics and the next highest percentage of incomplete trays were for spine surgery.

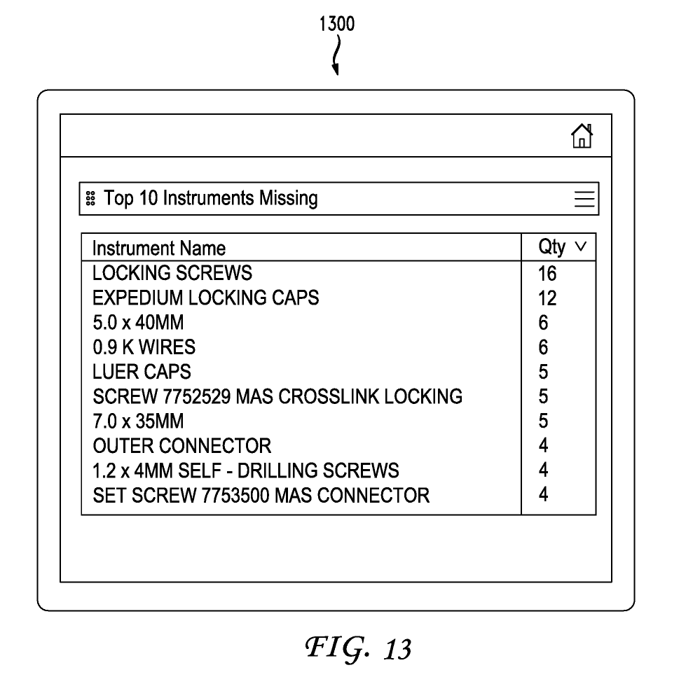

FIG. 13 illustrates a an example graphical-user interface that may be generated by the server computing device 370, referred to as graphic 1300 which identifies the top 10 instruments missing from trays. As can be seen, locking screws where the highest quantity of instruments missing followed by expedium locking caps. With respect to FIG. 13, the information provided in this figure illustrates the type of information that can be utilized by the algorithms disclosed herein. For example, where the system knows that locking screws are the most likely instrument to be missing, this data can be incorporated into the algorithms where probabilities are determined regarding a likelihood of success to have a full instrument tray that is sterilized ready for a particular surgery. Thus, if one instrument tray includes locking screws, the likelihood of issues with respect to that instrument tray will increase because locking screws are the top instrument that goes missing.

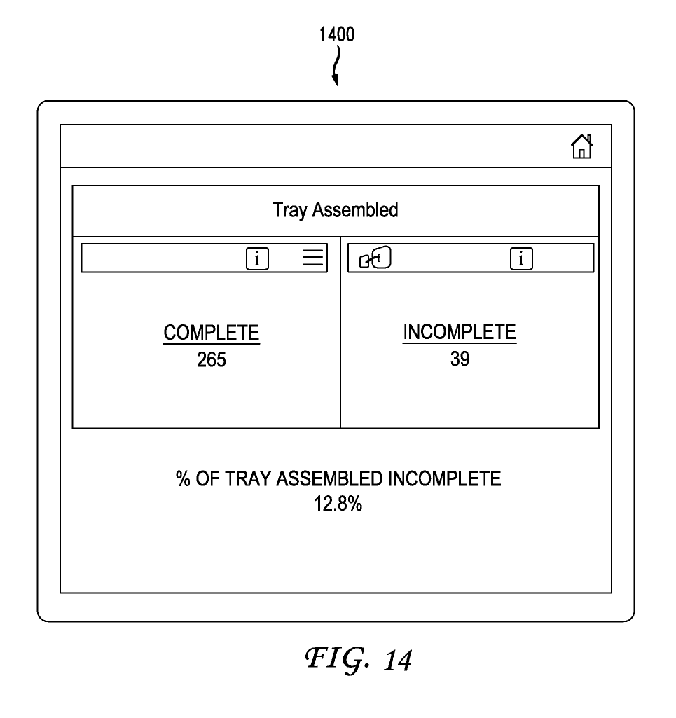

FIG. 14 illustrates an example graphical-user interface that may be generated by the server computing device 370, referred to as a dashboard graphic 1400 that shows the number of trays assembled and includes a listing of complete trays as well as incomplete trays with a calculation of the percentage of trays that are assembled incompletely. Again, this type of information can be utilized when the system assesses probabilities of success for particular trays.

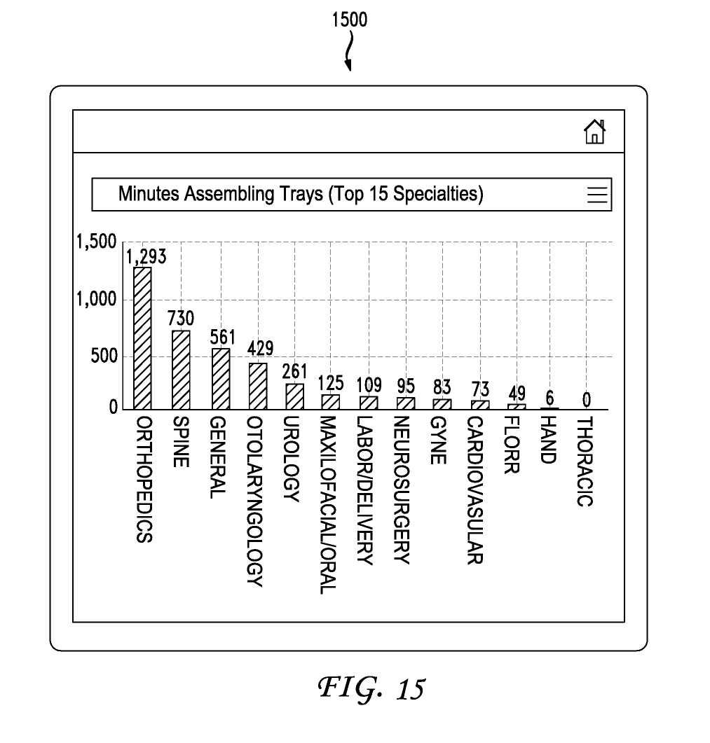

FIG. 15 illustrates an example graphical-user interface that may be generated by the server computing device 370, referred to as a dashboard interface 1500 that includes analysis of the minutes assembling trays broken down by specialty. The system utilizes all of the data received for how long it takes to assemble particular instrument trays and organizes that information into a particular graphic which can be utilized and viewed by staff members.

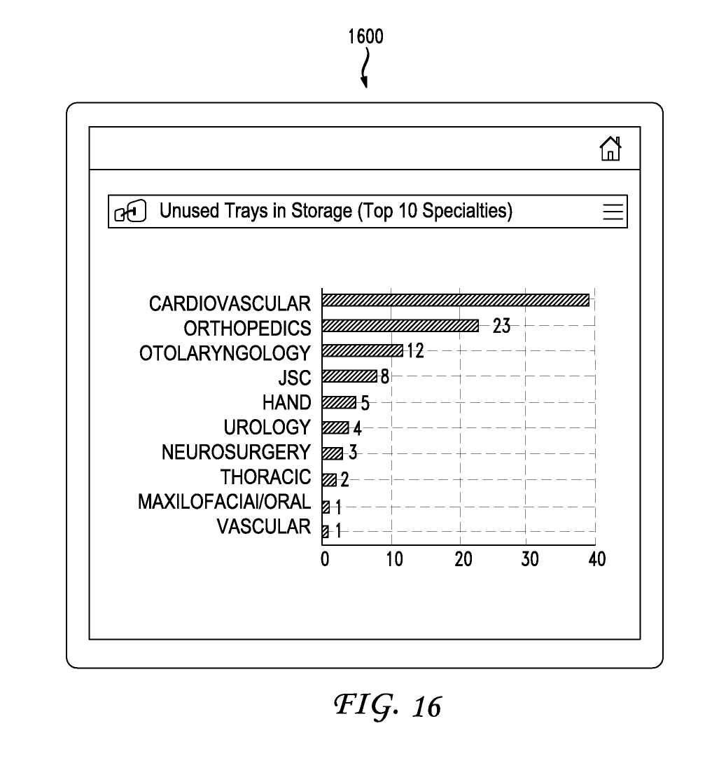

FIG. 16 illustrates an example graphical-user interface that can be generated by the server computing device 370, referred to as graphic 1600 that identifies, according to speciality, how many unused trays are in storage. As can be seen in the figure, cardiovascular trays have the highest number of unused trays in the storage followed by orthopedics.

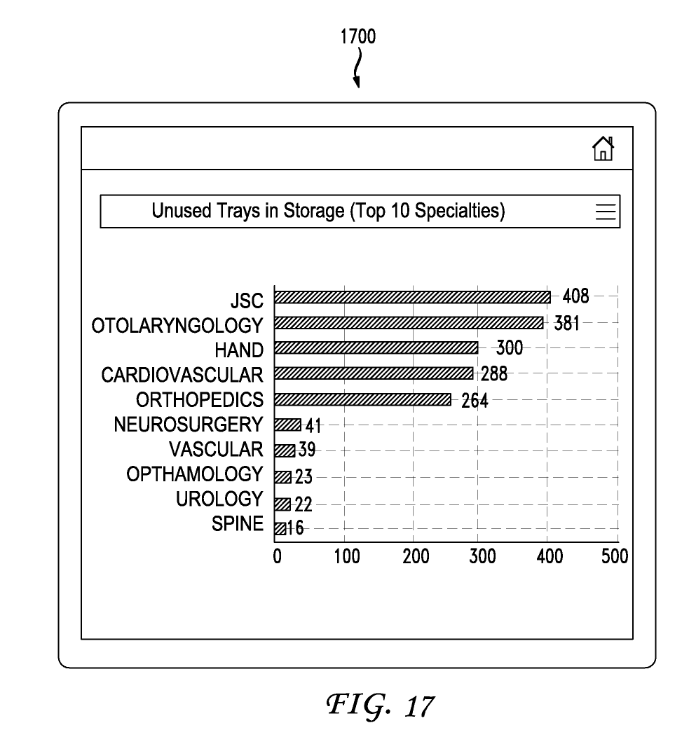

FIG. 17 illustrates an example graphical-user interface that can be generated by the server computing device 370, referred to as a dashboard graphic 1700 that shows unused instruments in storage based on specialty. This lists which individual instruments are not used and in storage to provide this useful information.

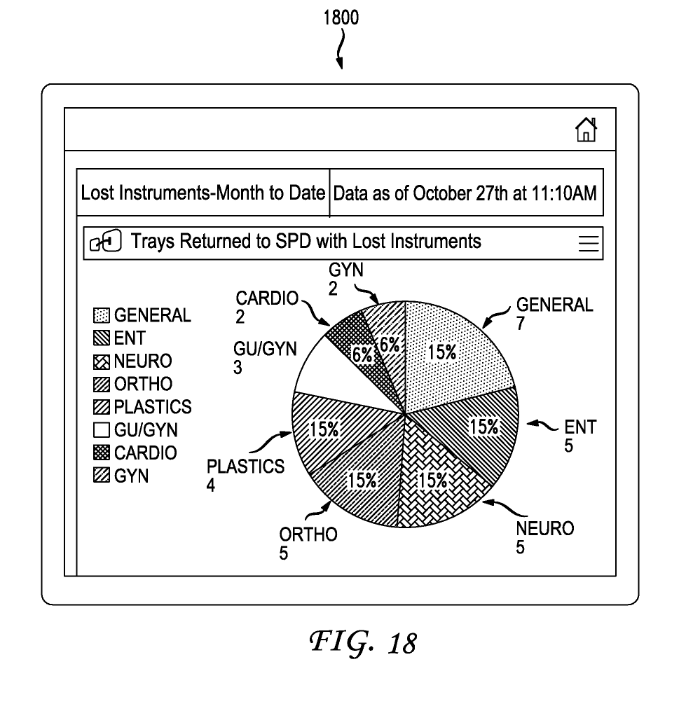

FIG. 18. Illustrates an example graphical-user interface that can be generated by the server computing device 370, referred to as dashboard view 1800 that shows the number of lost instruments according to a particular period of time such as month to date. The graphic 1800 illustrates a listing of trays returns to the SPD 122 with lost instruments. This can be broken out also by special specialty such as cardiovascular, orthopedic, and so on.

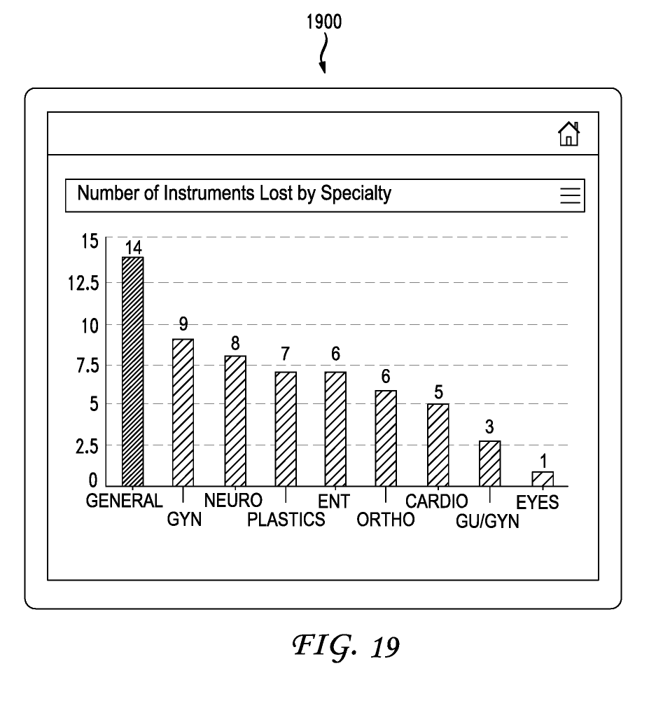

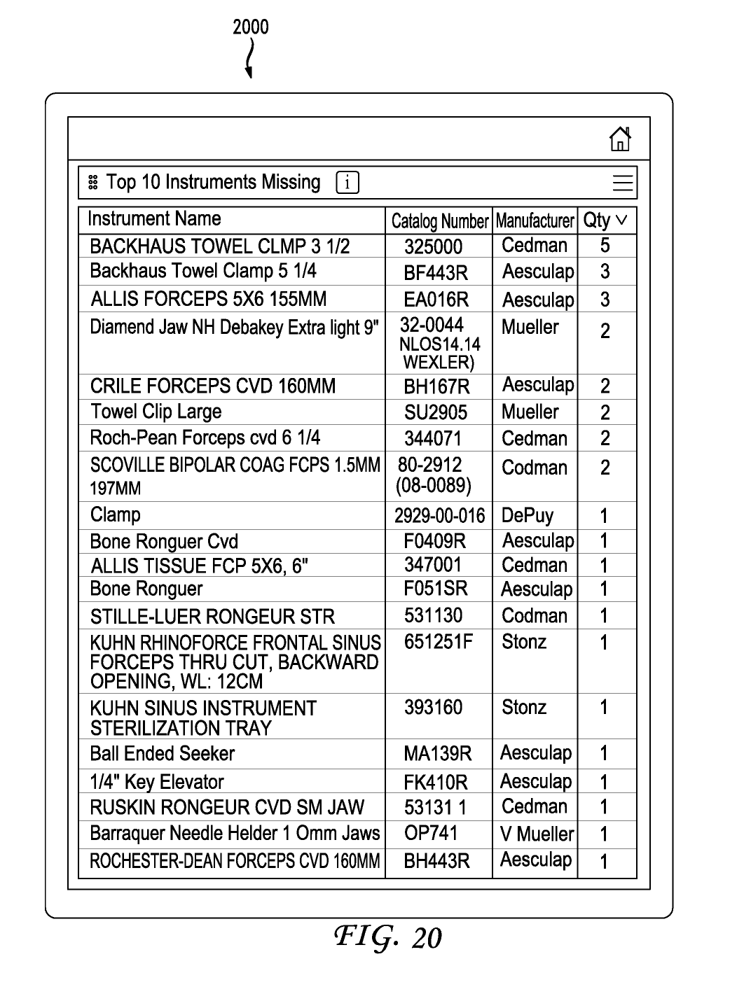

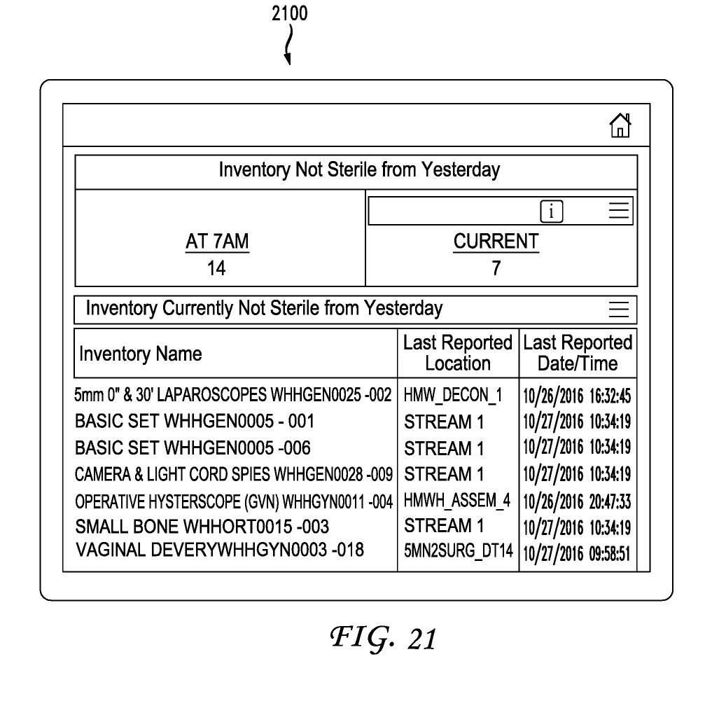

FIG. 19 illustrates an example graphical-user interface that can be generated by the server computing device 370, referred to as dashboard view of interface 1900 that lists a number of instruments lost by specialty. FIG. 20 illustrates a graphical interface 2000 listing the top 20 instruments lost including catalog number, manufacturer, instrument name, and the quantity lost. Other interfaces of course can show instruments lost in the OR department by total as well as trays assembled incomplete due to those lost instruments. FIG. 21 illustrates a dashboard graphical interface 2100 that includes a listing of inventory not sterile from a previous day or based on the current time. For example, the example interface shows that at 7 AM that morning, 14 units of inventory were not sterile and at the current time seven are not sterile. This report can include a current inventory name, last reported location, and last reported time.

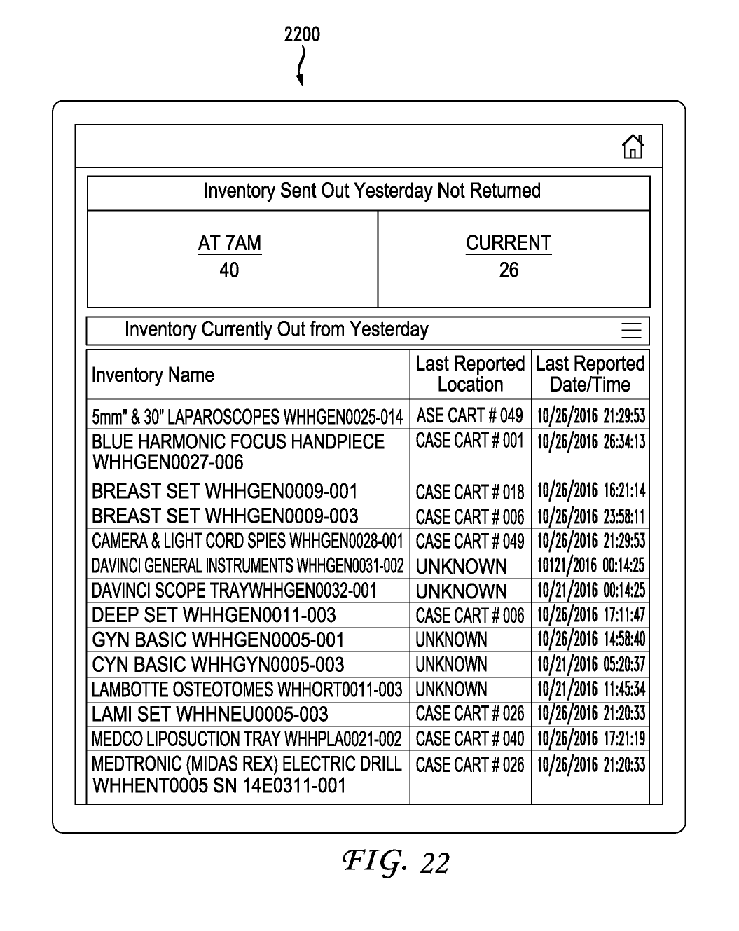

FIG. 22 illustrates an example graphical-user interface that can be generated by the server computing device 370, referred to as interface 2200, which identifies inventory sent out yesterday but not returned. Again this can also include an exemplary particular period of time such as inventory sent out at 7 AM that is not returned at 40 and the current number of inventory sent out but not report turned at 26. This can also include listings of inventory name, last reported location, and last reported date or time.

Another aspect of this disclosure relates to using machine learning algorithms to evaluate and analyze all of the various pieces of data associated with surgical instrument management including tracking where individual staff members go and the patterns of movement. Furthermore, it is typically only a particular number of places where surgical instruments or employees go. For example, surgical trays are either in sterilizers, storage facilities awaiting transportation up or down elevator to an operating room, and so forth. Individual employees typically work either in the operating room, the SPD, or other standardized locations. The system, using the RFID's on surgical trays and employees, can track and automatically learn the patterns of movements of individual people as well as trays. The analysis will look at the historical information of such movements, the timings of individuals entering rooms leaving rooms and working at particular computer stations, and so forth.

The disclosed computerized data collection system produces metadata that is specific to the generation of predictive analysis focused on resources required to perform surgical procedures on patients. Surgical case delays are major source of cost to the hospital and have a direct impact on the quality of patient care provided. Extensive delays in surgical case start times are an everyday patient care issue in hospital operating rooms around the world.

Delays in surgical case start times have a cascade effect that impacts management's ability to make efficient room assignments, Postoperative Acute Care Unit compression caused by delayed cases building up at the end of the day, and staffing overlaps that create scenarios where specific skilled nursing is not available for specific surgical procedures.

The systems disclosed herein model the impact of the mismatch of the scheduling to sterile processing resources by utilizing algorithms and business rules within the computerized data collection system. The mismatch of resources includes instrument trays and staff. Both resources are tracked and analyzed by machine learning algorithms.

All of this information can be utilized to tailor the communication, notices, structure of messages with selectable objects, and/or decisions made by the system disclosed herein. For example, staff predictability is an important component in terms of efficiently managing surgical instrument trays. Using the RFID technology disclosed herein, the system can track the movement of staff members wearing an RFID tag. Movement based on the location of their mobile device can also be used. There may be only five exits out of a particular area and the system can determine, for example, that over time once a particular employee exits a particular door, he does not return for several hours. This type of information is fed into the system, and when an alert or trigger is generated such that a particular instrument tray is needed immediately for a patient with a bullet wound, the system can trigger a particular alert which, according to a given time, may be provided for that particular employee because they had the most recent experience preparing the necessary instrument tray. However, if the trigger occurs during the two hour period of time in which is it is expected that the particular employee is not in the SPD 122, then the system could utilize that information and cause the alert to go to an alternate employee, supervisor, or some other identified individual. Further, the particular notice may also be adjusted to accommodate for that circumstance. In other words, if that particular employee was known to be in the SPD, the normal alert may be sent. However, if the particular employee is expected to be out of the hospital during that time, or detected to be out of the hospital, the not only can a normal alert be sent through the management application, but a separate text may be provided to the particular individual notifying them of the urgent need to return to manage the instrument tray.

Furthermore, a specific notice may be provided to a managing nurse of the circumstance with a pre-configured object for that particular employee's phone number, such that the nurse might be able to simply interact with the object to call that particular employee to find where he or she is and to immediately prepare the necessary tray. This provides one specific example of a dynamically generated message with particular functionality presented in the object. Again, assume that a particular employee is the preferred individual to prepare an instrument tray. However, given the data that the system has, the system performs an analysis which predicts that the likelihood is that the individual is off-site. A message can be presented to the supervisor which includes a configured object which, when interacted with by the manager, initiates the functionality of a telephone call to the appropriate individual. In other circumstances, if the timeframe is different and the system identifies that that individual is in the SPD, then no such button or object is presented to a manager and a normal message or notification can be presented instructing that individual to process the particular tray to prepare for surgery.

It is noted in one aspect that the RFID on individual staff members as well as individual trays can be used to track all of these items in the system. Other technologies, if available, could also be utilized to identify locations of trays and or people. Wi-Fi, cellular networks, GPS coordinates, and so forth may or may not be available in hospital environments. However, this disclosure encompasses any location-based technology which can be used to track individuals or trays.

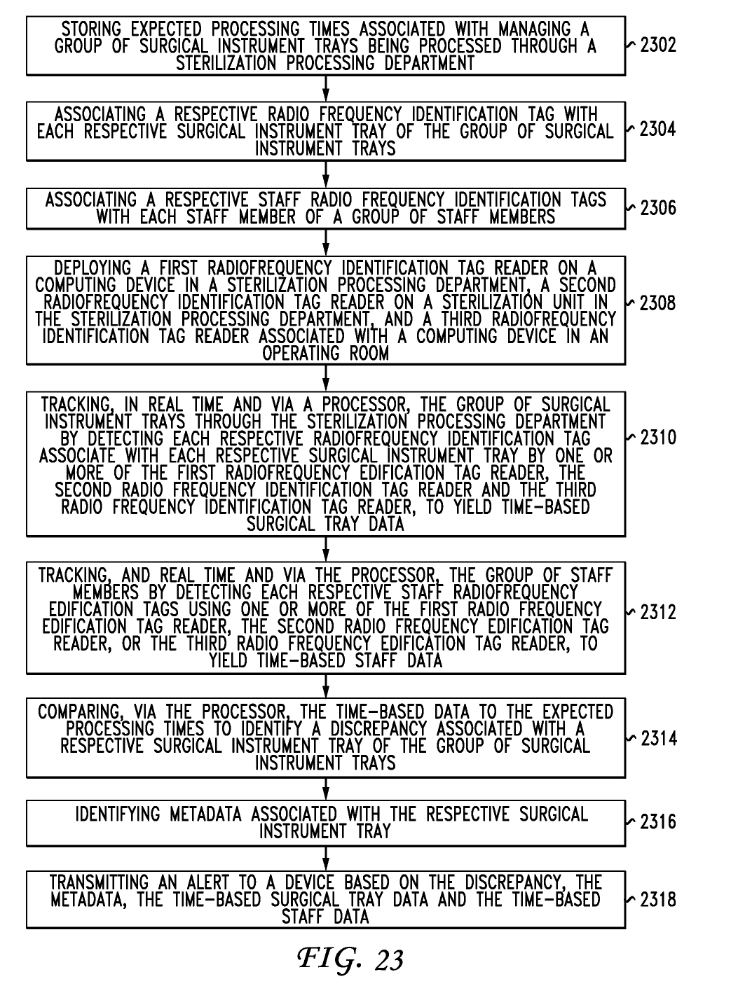

FIG. 23 illustrates a method aspect related to the use of RFIDs. The example method includes storing expected processing times associated with managing a group of surgical instrument trays being processed through a sterilization processing department (2302), associating a respective radio frequency identification tag with each respective surgical instrument tray of the group of surgical instrument trays (2304), associating a respective staff radio frequency identification tags with each staff member of a group of staff members (2306), deploying a first radiofrequency identification tag reader on a computing device in a sterilization processing department, a second radiofrequency identification tag reader on a sterilization unit in the sterilization processing department, and a third radiofrequency identification tag reader associated with a computing device in an operating room (2308), tracking, in real-time and via a processor, the group of surgical instrument trays through the sterilization processing department by detecting each respective radiofrequency identification tag associate with each respective surgical instrument tray by one or more of the first radiofrequency edification tag reader, the second radio frequency identification tag reader and the third radio frequency identification tag reader, to yield time-based surgical tray data (2310), tracking, in real-time and using the server computing device 370, the group of staff members by detecting each respective staff radiofrequency edification tag using one or more of the first radio frequency edification tag reader, the second radio frequency edification tag reader, or the third radio frequency edification tag reader, to yield time-based staff data (2312), comparing, using the server computing device 370, the time-based data to the expected processing times to identify a discrepancy associated with a respective surgical instrument tray of the group of surgical instrument trays (2314), identifying metadata associated with the respective surgical instrument tray (2316) and transmitting an alert to a device based on the discrepancy, the metadata, the time-based surgical tray data and the time-based staff data (2318).

Other aspects of this embodiment include receiving an instruction from a user of the device and, based on the instruction, identifying a responsible person for the respective surgical instrument tray and transmitting a message to a second device associated with the responsible person that instructs the responsible person to prioritize a processing of the respective surgical instrument tray.

Intelligence Scheduling

Another aspect of this disclosure relates to a scheduling engine that utilizes all the intelligence gained through the historical data that is available because of the system disclosed herein. Typically, doctors or hospitals will schedule surgical procedures in an operating room and based on that schedule, those managing the surgical instrument trays will seek to prepare and have ready sterile surgical instruments for the procedure. The scheduling does not take into account at all the status of or expected availability of surgical instruments according to historical usage patterns or any kind of predictive technology. Inasmuch as the system disclosed herein retains and stores historical movement patterns, gap analyses, employee analyses, data on availability of surgical instrument trays and historical turnaround times, and so forth (any of the data disclosed herein about the process or a component of the process), an aspect of this disclosure is to utilize one or more parameters gained through an analysis of the cycle of use for individual surgical trays to affect the scheduling of operating rooms for surgical procedures. In some aspects, the effects may be at different levels. Thresholds can be set as well to implement the type of input available from knowledge of the management of trays of surgical instruments. For example, if a doctor seeks to schedule a surgical procedure in an operating room at 2 PM on Wednesday, the system can perform an analysis of the probability of successfully having the necessary surgical instrument tray available for the surgery at the right time. Assume that given the knowledge of the management of surgical instrument trays by the system, that historically there is a 5% increase in the probability of an issue with the surgical tray at 2 PM whereas a higher probability of successfully receiving a sterile surgical tray exists at 11 AM. In other words, the system may know that there is a 98% probability of success at 11 AM and only a 93% probability of success at 2 PM. The system could provide a suggestion that a better time for the operation would be at 11 AM based on the surgical instrument tray management analysis. Of course a doctor or other staff member can override the suggestion but the system can provide the option which can improve the health of the patient. The system can also evaluate in its analysis any number of parameters in making this suggestion. For example, the system can evaluate any one or more of the type of surgery, characteristics of the patient, characteristics of the doctor or staff, staff members in the SPD 122 that would likely be utilized to prepare a surgical instrument tray given the proposed time for the surgery, data about the instruments and whether they may be loaner instruments, or native instruments to the hospital, knowledge of the schedule of all of the other instrument trays and their expected availability, historical information associated with the time of day, the particular instrument tray needed, the current schedule of surgical procedures, predictive components of what would be expected in the future by way of surgical instrument processing, data about other hospitals in the potential availability of loaner trays, data about which instruments are most often missing from instrument trays and whether that affects the particular tray needed for a surgery to be scheduled, and so forth. Any one or more of these parameters can be evaluated and analyzed to arrive at a suggestion of potentially a different time for a surgical procedure.

In one aspect, the level of suggestiveness that the system provides can depend on a probability value associated with a successful delivery of the surgical instruments for the surgery. For example, if a requested surgical time has a corresponding probability of success of over 90%, the system may provide no suggestion. However, if the requested surgical time only has an 80% probability of success, then the system may provide a suggestion of a better time, but have an easy or a threshold for overriding the suggestion. However, if the requested time only has a 60% probability of the surgical instruments being available, then the system may prevent the user from scheduling the surgery at that time and force them into a new time that has at least an 85% probability of success. Thus, the system could provide a sliding scale which balances how much to force the schedule relative to the probability of success for a requested time for surgery.

In another aspect, the system could provide an interface where the user inputs characteristics of the surgery, an identification of which can yield a knowledge of what surgical instruments are needed. The system can obtain any data necessary or helpful for the system to use, and simply receive proposed timeslots for the surgical procedure. From these proposed timeslots which provide optional slots that have a high probability of success, the user could simply select the appropriate timeslot to schedule the surgery. In another example, the user may request that only timeslots be presented which represent a 95% or greater probability of successfully receiving the proper instrument tray. Then, if the appropriate time does not fit with the patient's schedule, the user may be able to manually adjust the threshold to receive more available times. As can be appreciated, by incorporating the knowledge of the system regarding all of the data points associated with surgical instrument tray management, and evaluating that data to arrive at predicted success rates for timely deliverance of sterile surgical instrument trays, the system can suggest or control scheduling of particular surgical procedures to enhance the probability of success with respect to surgical instrument tray deliverance and thus increase patient healthcare.

In another aspect, scheduling surgical procedures often use “preference cards” which enable doctors to include preferences for times that they desire. Often, these cards are incomplete and not kept up to date. The preference cards are supposed to be used for managing conflicts between different doctor preferences. However, often such preferences are ignored and surgical schedule times are just set. The system disclosed herein, utilizing the knowledge of all of the surgical instrument inventory, and a real-time status of the inventory, as well as insight into projected or predicted availability of particular instruments or instrument trays, as well as other parameters, can be inserted into the scheduling process and help to resolve conflicts, and provide input at any level into the scheduling process.

The knowledge can also include the “block time” associated with surgeons having their own down time or certain blocks of time that the surgeon is going to do back-to-back cases. Thus, if a surgeon is known to want to schedule five surgical procedures on a Tuesday morning, back to back, the scheduling of those procedures can take into account the knowledge of the system has of the inventory needed for those five surgeries. The system can perform its gap analysis with respect to how long it's going to take to process the individual surgical trays and can aid the scheduling process by perhaps ordering those five surgeries in a particular order so as to minimize error in delivering properly sterilized trays for each individual case.

In another aspect, practically speaking, scheduling always has last-minute changes and adjustments. For example, one person might be scheduled for surgery on one day, but the blood pressure was high or the enzymes were off and the surgeon decided at the last minute not to perform the surgery and check the next day. These kinds of changes often never get into the scheduler. The system disclosed herein can receive data regarding the cancellation of a procedure, or the scheduling of a last-minute procedure, and process that information to adjust gap times in processing a particular tray, transmit particular notices to employees to accelerate the sterilization for a particular tray, or reduce the priority of a tray such that other trays can now be processed sooner. All of these changes can be managed by the present system such that unexpected adjustments can easily not only affect individual instruments that are directly related to the change in schedule but improve the processing in other instruments as well given the changes. In one example, if a surgeon desires to insert somebody into the schedule that was unexpected, a query could be made to the system asking about how quickly the instrument tray for that particular procedure could be prepared on a priority basis. The system could respond with a number such as three hours. In this scenario, the surgeon to schedule the surgery for three hours away with confidence that the surgical instruments needed will be delivered and sterile. The knowledge of the system has of the overall SPD process eliminates the need of manual phone calls down to the SPD 122 asking for somebody to search around for the property instruments to see if they are ready for surgery now.

In one aspect, the system could provide a confirming notification to a staff member regarding the surgery. For example, 15 minutes before scheduled surgery (or any chosen timeframe), a confirming communication can be sent through the system such that the head nurse or doctor can confirm that the surgery is going to take place. If it is not, an interface can be provided where the rescheduled time, or circumstances, can be submitted. The particularly defined interface enables the system to quickly gather the information regarding the rescheduled surgery, which information can then be utilized by the algorithms disclosed herein for evaluating and analyzing the surgical instrument tray processes. In another aspect, if a staff member indicates that the surgery has been rescheduled, the system can evaluate or access the hospital scheduler and identify if that patient has already been rescheduled. In such case, the data in the database will be modified to accommodate this revised schedule. The system disclosed herein can, in real time or virtually in real time, incorporate such changes into the overall algorithm and overall of valuation of the processing of surgical instrument trays.

In another aspect, surgeons may include on their preference card a list of the five trays of instruments that they need. These are called in the industry “have availables” which can include a series of instruments that they may or may not ever use. In some cases, 25% of the inventory is this type of instrument. This can include some instruments that the surgeon perhaps used some years ago and just wants to have available in case they may need it. The present system can also provide an analysis of the “have available” instruments which can aid a surgeon in perhaps making more intelligent decisions regarding those instruments. For example, upon receiving the preference card, the system could present the probability that each additional tray of instruments would be used in this particular surgery. For example, one of the desirable trays of instruments may not have been used for 15 years. If the surgeon is reminded of that historical information, she may drop that request from the preference card. In this regard, an aspect of this disclosure involves engaging in an interactive interface with the surgeon with respect to preference cards in order to improve the listing of surgical instruments which are to be made available for a particular surgery. Fine tuning this list can alleviate the burden of making available all of the desired instruments for a particular surgery. In other words, if one instruments set which is designated as “make available” has such a low probability of being used that the system can persuade the surgeon simply not to list that set in the “make available” list, this can alleviate some pressure on the system.

In some cases, there is “dormant” inventory as well that are in storage that has not been used for a long time. The present software can also provide a summary of such instrumentation and let the proper staff know of the list of dormant trays that have not been touched in a year, or whatever timeframe is desirable.

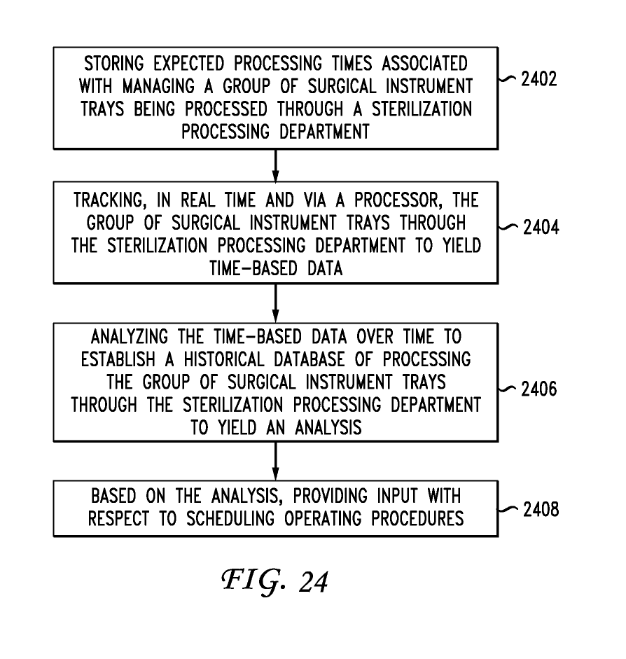

FIG. 24 illustrates an example method related to intelligence scheduling of surgeries using inventory processing data. An example method includes storing expected processing times associated with managing a group of surgical instrument trays being processed through a sterilization processing department (2402), tracking, in real-time and via a processor, the group of surgical instrument trays through the sterilization processing department to yield time-based data (2404), analyzing the time-based data over time to establish a historical database of processing the group of surgical instrument trays through the sterilization processing department to yield an analysis (2406) and, based on the analysis, providing input with respect to scheduling operating procedures (2408).

The input can include suggesting a time to schedule a respective surgical operation such that a chosen surgical instrument tray has a probability above a threshold value of being prepared, sterile, and delivered for the respective surgical operation. The level of input can vary as well. For example, the input can provide a suggested time or block of time on a scheduling interface for a person scheduling a surgery. The input could also include more strident control of the schedule such that, according to a certain threshold, a person scheduling the surgery is not allowed to schedule the surgery in a timeslot in which the probability of successfully receiving sterile instruments is below 90% (or any other threshold value).

Global ID for Instrument Trays

In another aspect, there are trays all across the world at different hospitals or in different facilities. One aspect of this disclosure is a concept using a Global ID for each tray. In this respect, whether it is nationwide or worldwide, each tray can have a unique ID. This is currently not done in the industry as identifiers are usually associated with the manufacturer of the instrument. In a system as is disclosed herein, it is desirable to have a unique identifier for every tray in the field. A clearinghouse could be established where all labeling of trays goes to the clearinghouse such that the system generates a label for a particular tray but the clearinghouse ensures that it is a new and unique number for that tray globally. In other words, there won't be a first manufacturer with the tray numbered 00112 as well as a second manufacturer with the tray having the same ID number of 00112.

Now, while the general concept of providing an ID to a surgical incident tray is not unique, a unique concept disclosed herein is how one may process a group of trays in such a way as to distinguish and identify trays that have duplicate numbers across hospitals, the manufacturers, and so on. The process would include storing individual tray IDs across hospitals and manufacturers and then processing all or a large pool of trays and identifying those which have unique tray ID numbers. Assume that 75% of the trays within a given hospital already have unique ID numbers given the universal pool of data. The system then would generate new unique ID numbers globally for the 25% of the trays that need them. The process can then proceed from hospital to hospital until the entire pool of trays each has a unique tray ID that is globally determined to be unique. Then, as new trays are added to the system, the global pool of unique tray IDs can be accessed and utilized such that each new tray is given a globally unique tray ID.

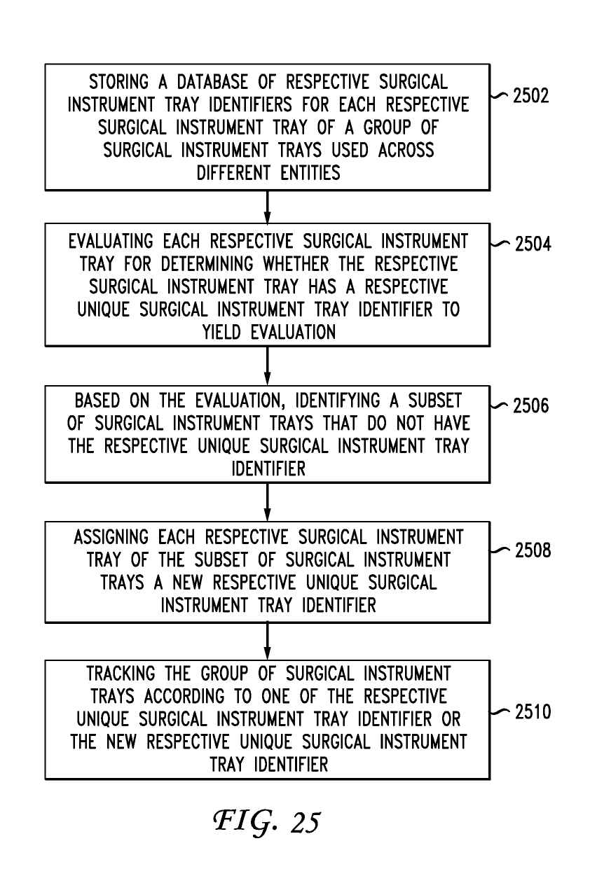

FIG. 25 illustrates a method embodiment related to global IDs for surgical instrument trays. The example method includes storing a database of respective surgical instrument tray identifiers for each respective surgical instrument tray of a group of surgical instrument trays used across different entities (2502), evaluating each respective surgical instrument tray for determining whether the respective surgical instrument tray has a respective unique surgical instrument tray identifier to yield evaluation (2504), based on the evaluation, identifying a subset of surgical instrument trays that do not have the respective unique surgical instrument tray identifier (2506), assigning each respective surgical instrument tray of the subset of surgical instrument trays a new respective unique surgical instrument tray identifier (2508) and tracking the group of surgical instrument trays according to one of the respective unique surgical instrument tray identifier or the new respective unique surgical instrument tray identifier (2510).

An important facet of using global IDs relates to patient care. If there is an issue with the patient the passes away in the operating room, and the tray is a loaner tray, without a global ID, it becomes very difficult to figure out the historical use of that tray. With a global ID, a central tracking entity can quickly identify who previously had surgery using instruments from that tray and who is scheduled in the future for surgery. Notices can be generated by the system and sent out to the proper individuals to highlight that there is a potential issue with the sterilization of that tray, or other issues such as a missing instrument or any other issued disclosed herein, such that precautions can be taken and infection control can be notified.

Another aspect of this disclosure involves providing a labeling service. An entity can maintain a central database of IDs associated with surgical instrument trays across multiple hospitals and potentially across multiple states and nations. In one scenario, assume that a particular hospital has a set of surgical instrument trays that it desires to have global IDs assigned to. That hospital can provide data to a central labeling entity which can process each individual tray and change its previous identification to a global permanent identification which can then be used, as noted above, to track that tray potentially across hospitals and more generally in its lifecycle. Such a tracking entity can track loaner trays as they are passed between hospitals in or from a global viewpoint.

Multiple Hospital Tray Management

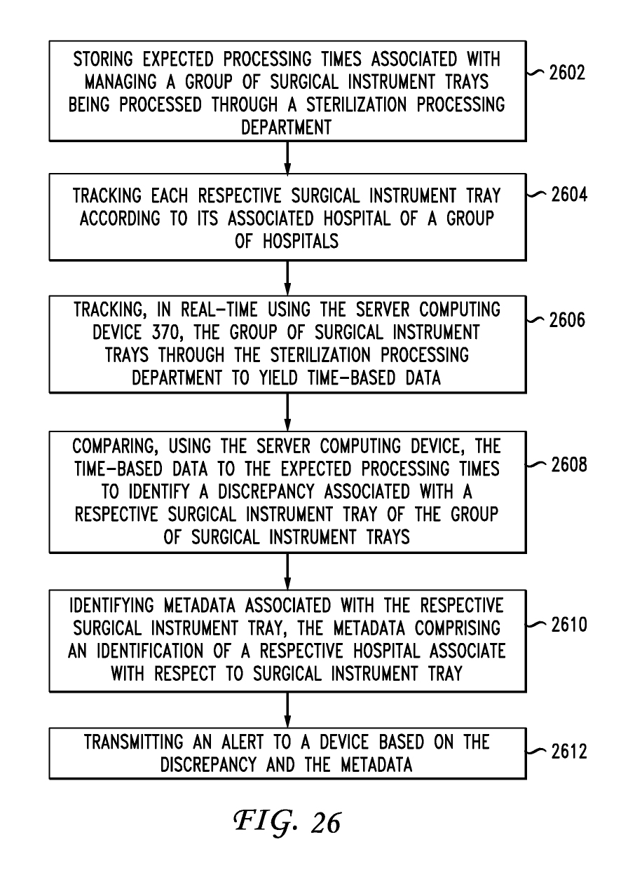

Another aspect of this disclosure relates to managing trays across multiple hospitals. In this respect, an example method is disclosed in FIG. 26. The example method includes storing expected processing times associated with managing a group of surgical instrument trays being processed through a sterilization processing department (2602), tracking each respective surgical instrument tray according to its associated hospital of a group of hospitals (2604), tracking, in real-time using the server computing device 370, the group of surgical instrument trays through the sterilization processing department to yield time-based data (2606), comparing, using the server computing device 370, the time-based data to the expected processing times to identify a discrepancy associated with a respective surgical instrument tray of the group of surgical instrument trays (2608), identifying metadata associated with the respective surgical instrument tray, the metadata comprising an identification of a respective hospital associate with respect to surgical instrument tray (2608) and transmitting an alert to a device based on the discrepancy and the metadata (2610).

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Those of skill in the art will appreciate that other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

The various embodiments described above are provided by way of illustration only and should not be construed to limit the scope of the disclosure. For example, the principles herein pertain generally to turn-taking for interactive spoken dialog systems, and are not constrained to a particular device or mechanism. Those skilled in the art will readily recognize various modifications and changes that may be made to the principles described herein without following the example embodiments and applications illustrated and described herein, and without departing from the spirit and scope of the disclosure.

We claim:

1. A method comprising:
storing expected processing times associated with managing a group of surgical instrument trays being processed through a sterilization processing department, the expected processing times comprising at least a first expected processing time associated with a first portion of processing the group of surgical instrument trays through the sterilization processing department and a second expected processing time associated with a second portion of processing the group of surgical instrument trays through the sterilization processing department;
tracking, in real-time and via a processor, the group of surgical instrument trays through the sterilization processing department to yield time-based data;
comparing, via the processor, the time-based data to the expected processing times to identify a discrepancy associated with a respective surgical instrument tray of the group of surgical instrument trays;
identifying metadata associated with the respective surgical instrument tray; and
transmitting an alert to a device based on the discrepancy and the metadata.

2. The method of claim 1, further comprising:
receiving an instruction from a user of the device;
based on the instruction, identifying a responsible person for the respective surgical instrument tray; and
transmitting a message to a second device associated with the responsible person that instructs the responsible person to prioritize a processing of the respective surgical instrument tray.

3. The method of claim 1, wherein the tracking in real-time comprises receiving tracking data for each surgical instrument tray of the group of surgical instrument trays based on an radio frequency identification tag physically associated with each surgical instrument tray.

4. The method of claim 1, wherein the discrepancy comprises a late check-in of a loaner surgical instrument tray.

5. The method of claim 1, where the discrepancy comprises a delay in a particular parameter associated with expected processing times.

6. The method of claim 5, where the particular parameter comprises one of: a late check-in time for a loaner surgical instrument tray, a storage time, a sterile processing time, a time associated with respective surgical tray being at a particular location, a loaner check-in in which an alert is sent to users notifying them of any loaner inventory checked in, a loaner checkout in which an alert is sent to users notifying them of any loaner inventory checked out, an inventory not sterile parameter in which an alert is sent to users when inventory without a sterilization record was sent to a location, a quick turnaround inventory marking, a sterilizer mismatch notification in which an alert is sent to users when an inventory item was scanned into a sterilization load with unapproved parameters, an incomplete tray in which an alert is sent to users when a tray was assembled incomplete, an inventory needs repair in which an alert is sent to users when an inventory item has reached a designated repair threshold, a busted assembly warning, a priority window override notice, and a tray marked quick turnaround is scanned into a sterilizer in which an alert is sent to update on any inventory previously identified quick turnaround inventory that has been scanned to the sterilization load.

7. The method of claim 1, wherein the metadata comprises at least one of: a manufacturer of the respective surgical instrument tray, the manufacturer of at least one instrument on the respective surgical instrument tray, a staff member who is experienced in processing the respective surgical instrument tray, a manager responsible for overseeing the processing of the respective surgical instrument tray, an individual who most recently prepared the respective surgical instrument tray, information about a patient scheduled to have surgery using the respective surgical instrument tray, information associated with a doctor scheduled to perform the surgery using the respective surgical instrument tray, information associated with a biological indicator that confirms sterile processing for the respective surgical instrument tray, parameters associated with a sterilization process for the respective surgical instrument tray, a priority associated with the respective surgical instrument tray, and a user history of the respective surgical instrument tray.

8. A method comprising:
obtaining, at a server computing device, expected time data corresponding to processing a group of surgical instrument trays in a sterilization process, the expected time data comprising a first expected time associated with the processing of a first portion of the group of surgical instrument trays and a second expected time associated with the processing of a second portion of the group of surgical instrument trays;
obtaining, in real-time, using the server computing device, actual time data corresponding to the processing of the group of surgical instrument trays;
comparing, using the server computing device, the actual time data to the first expected time and the second expected time to identify a discrepancy for a respective surgical instrument tray of the group of surgical instrument trays;
identifying, using the server computing device, metadata associated with the respective surgical instrument tray; and
transmitting, using the server computing device, a notification to a client device based on the discrepancy and the metadata.

9. The method of claim 8, further comprising:
dynamically generating the notification to comprise an object which, when interacted with by a user, causes a function to be performed which addresses the discrepancy.

10. The method of claim 8, wherein the obtaining the actual time data comprises:
accessing a respective radio frequency identification tag physically associated with each surgical instrument tray of the group of surgical instrument trays; and
obtaining data, from the respective radio frequency identification tag, for inclusion in the actual time data.

11. The method of claim 8, wherein transmitting the notification to the client device further comprises transmitting the notification to a group of client devices based on the metadata.

12. The method of claim 9, wherein the function performed when the object is interacted with comprises transmitting an alert to a second user with instructions to process the respective surgical instrument tray.

13. A system comprising:
a client device;
at least one processor; and
a computer readable storage device storing instructions which, when executed by the at least one processor, cause the at least one processor to perform operations comprising:
obtaining expected time data corresponding to processing a group of surgical instrument trays in a sterilization process, the expected time data comprising a first expected time associated with the processing of a first portion of the group of surgical instrument trays and a second expected time associated with the processing of a second portion of the group of surgical instrument trays;
obtaining actual time data corresponding to the processing of the group of surgical instrument trays;
comparing the actual time data to the first expected time and the second expected time to identify a discrepancy for a respective surgical instrument tray of the group of surgical instrument trays;
identifying metadata associated with the respective surgical instrument tray; and
transmitting a notification to the client device based on the discrepancy and the metadata.

14. The system of claim 13, wherein the computer readable storage device stores additional instructions which, when executed by the at least one processor, cause the at least one processor to perform further operations comprising:
dynamically generating the notification to comprise one or more objects which, when interacted with, cause a function to be performed which addresses the discrepancy.

15. The system of claim 13, further comprising:
a radio frequency identification reader, in operable communication with the at least one processor, to access a respective radio frequency identification tag physically associated with each surgical instrument tray of the group of surgical instrument trays; and
wherein the actual time data is obtained, by the at least one processor, from the radio frequency identification reader.

16. The system of claim 13, wherein the computer readable storage device stores additional instructions which, when executed by the at least one processor, cause the at least one processor to perform further operations comprising:
dynamically generating the notification to comprise an object which, when interacted with by a user, causes a function to be performed which addresses the discrepancy.

17. The system of claim 13, wherein transmitting the notification to the client device further comprises transmitting the notification to a group of client devices based on the metadata.

18. The system of claim 14, wherein the function performed when the object is interacted with comprises transmitting an alert to a second user with instructions to process the respective surgical instrument tray.

19. A non-transitory computer readable device encoded with instructions for managing surgical instruments, the instructions, when executed by a processor, cause the processor to perform operations comprising:
obtaining expected time data corresponding to processing a group of surgical instrument trays in a sterilization process, the expected time data comprising a first expected time associated with the processing of a first portion of the group of surgical instrument trays and a second expected time associated with the processing of a second portion of the group of surgical instrument trays;
obtaining, in real-time actual time data corresponding to the processing of the group of surgical instrument trays;
comparing the actual time data to the first expected time and the second expected time to identify a discrepancy for a respective surgical instrument tray of the group of surgical instrument trays;
identifying metadata associated with the respective surgical instrument tray; and
transmitting a notification to a client device based on the discrepancy and the metadata.

20. The non-transitory computer readable device of claim 19, wherein the obtaining of the actual time data comprises:
accessing a respective radio frequency identification tag physically associated with each surgical instrument tray of the group of surgical instrument trays; and
obtaining data, from the respective radio frequency identification tag, for inclusion in the actual time data.
